SEQUENCE LISTING (30 sequences)

SEQ ID NO: 1 - DNA, 20 bp, Artificial Sequence
  Synthetic gRNA
gcttttctacacacacccgg

SEQ ID NO: 2 - DNA, 20 bp, Artificial Sequence
  Synthetic gRNA
cacgtgcgatatgatcccgg

SEQ ID NO: 3 - DNA, 20 bp, Artificial Sequence
  Synthetic gRNA
ggagtccacacacaacacca

SEQ ID NO: 4 - DNA, 20 bp, Artificial Sequence
  Synthetic gRNA
tactcgtccatagaccacgc

SEQ ID NO: 5 - DNA, 73 bp, Artificial Sequence
  Synthetic Primer
gcggcccgggttcgattcccggccgatgcagcttttctacacacacccgggttttagagctagaaatagcaag

SEQ ID NO: 6 - DNA, 43 bp, Artificial Sequence
  Synthetic Primer
ccgggatcatatcgcacgtgtgcaccagccgggaatcgaaccc

SEQ ID NO: 7 - DNA, 43 bp, Artificial Sequence
  Synthetic Primer
cacgtgcgatatgatcccgggttttagagctagaaatagcaag

SEQ ID NO: 8 - DNA, 43 bp, Artificial Sequence
  Synthetic Primer
tggtgttgtgtgtggactcctgcaccagccgggaatcgaaccc

SEQ ID NO: 9 - DNA, 43 bp, Artificial Sequence
  Synthetic Primer
ggagtccacacacaacaccagttttagagctagaaatagcaag

SEQ ID NO: 10 - DNA, 73 bp, Artificial Sequence
  Synthetic Primer
attttaacttgctatttctagctctaaaacgcgtggtctatggacgagtatgcaccagccgggaatcgaaccc

SEQ ID NO: 11 - DNA, 20 bp, Artificial Sequence
  Synthetic gRNA
gtgtccccttattcgtgcgg

SEQ ID NO: 12 - DNA, 20 bp, Artificial Sequence
  Synthetic gRNA
cacactatcaaaggatgacg

SEQ ID NO: 13 - DNA, 20 bp, Artificial Sequence
  Synthetic gRNA
ataaggggacacactatcaa

SEQ ID NO: 14 - DNA, 20 bp, Artificial Sequence
  Synthetic gRNA
agtgtgtccccttattcgtg

SEQ ID NO: 15 - DNA, 73 bp, Artificial Sequence
  Synthetic Primer
gcggcccgggttcgattcccggccgatgcagtgtccccttattcgtgcgggttttagagctagaaatagcaag

SEQ ID NO: 16 - DNA, 43 bp, Artificial Sequence
  Synthetic Primer
cgtcatcctttgatagtgtgtgcaccagccgggaatcgaaccc

SEQ ID NO: 17 - DNA, 43 bp, Artificial Sequence
  Synthetic Primer
cacactatcaaaggatgacggttttagagctagaaatagcaag

SEQ ID NO: 18 - DNA, 43 bp, Artificial Sequence
  Synthetic Primer
ttgatagtgtgtccccttattgcaccagccgggaatcgaaccc

SEQ ID NO: 19 - DNA, 43 bp, Artificial Sequence
  Synthetic Primer
ataaggggacacactatcaagttttagagctagaaatagcaag

SEQ ID NO: 20 - DNA, 73 bp, Artificial Sequence
  Synthetic Primer
attttaacttgctatttctagctctaaaaccacgaataaggggacacacttgcaccagccgggaatcgaaccc

SEQ ID NO: 21 - DNA, 21 bp, Artificial Sequence
  Synthetic Primer
tttgactacgcggactcattc

SEQ ID NO: 22 - DNA, 22 bp, Artificial Sequence
  Synthetic Primer
cgcggtattgcatattggactt

SEQ ID NO: 23 - DNA, 21 bp, Artificial Sequence
  Synthetic Primer
aacacagtctttaggccagca

SEQ ID NO: 24 - DNA, 21 bp, Artificial Sequence
  Synthetic Primer
ccgtgaggtagagttcaaccg

SEQ ID NO: 25 - DNA, 21 bp, Artificial Sequence
  Synthetic Primer
acaaacgtcgctgatccacta

SEQ ID NO: 26 - DNA, 19 bp, Artificial Sequence
  Synthetic Primer
ggaagacacacgactcggc

SEQ ID NO: 27 - DNA, 20 bp, Artificial Sequence
  Synthetic Primer
ggttgctacgtccttctgga

SEQ ID NO: 28 - DNA, 20 bp, Artificial Sequence
  Synthetic Primer
ggttgctacgtccttctgga

SEQ ID NO: 29 - DNA, 21 bp, Artificial Sequence
  Synthetic Primer
ttgtctgggcaagaggatcag

SEQ ID NO: 30 - DNA, 21 bp, Artificial Sequence
  Synthetic Primer
accactcgcacttgcactttc

(19) United States
(12) Patent Application Publication
(10) Pub. No.: US 2021/0161937 A1
(43) Pub. Date: Jun. 3, 2021

(54) ACTIVATION OF MITOCHONDRIAL UNCOUPLING AS A THERAPEUTIC INTERVENTION

(71) Applicant: The Trustees of Columbia University in the City of New York, New York, NY (US)

(72) Inventors: Mimi Shirasu-Hiza, New York, NY (US); Julie Canman, New York, NY (US); Matt Ulgherait, New York, NY (US)

(21) Appl. No.: 16/961,767

(22) PCT Filed: Jan. 23, 2019
(86) PCT No.: PCT/US2019/014689

§ 371 Date: Jul. 13, 2020

Related U.S. Application Data
(60) Provisional application No. 62/620,621, filed on Jan. 23, 2018.

(51) Int. Cl. A61K 31/7088; A61K 45/06; A61K 31/567; A61K 31/20; A61K 31/06; A61K 31/05; A61K 31/167; A61K 31/426; A61K 31/4985; A61P 35/00

(52) U.S. Cl. CPC A61K 31/7088; A61K 45/06; A61K 31/567; A61K 31/20; A61P 35/00; A61K 31/05; A61K 31/167; A61K 31/426; A61K 31/4985; A61K 31/06

(57) ABSTRACT

The present invention relates to activating mitochondrial uncoupling as a therapeutic intervention which has many beneficial effects to both healthy and sick subjects. The beneficial effects of activating mitochondrial uncoupling include extended lifespan, decreasing or suppressing harmful or unwanted cellular proliferation, increasing or restoring intestinal homeostasis and slowing or inhibiting tumor growth. The invention includes the administration of activators of mitochondrial uncoupling to subjects in order to extend lifespan, decrease or suppress harmful or unwanted cellular proliferation, increase or restore intestinal homeostasis, prevent and treat cancer, and slow or inhibit tumor growth.

### CROSS REFERENCE TO RELATED APPLICATION

The present application claims priority to U.S. patent application Ser. No. 62/620,621 filed Jan. 23, 2018, which is hereby incorporated by reference in its entirety.

This invention was made with government support under through grants 5T32DK007328, 5T32HL120826, F31GM125363, R01GM117407, R35GM127049, R35GM124717, R21DK112074, and R01AG045842, all awarded by NIH. The government has certain rights in the invention.

### FIELD OF THE INVENTION

The present invention relates to activating mitochondrial uncoupling as a therapeutic intervention which has many beneficial effects to both healthy and sick subjects. The effects of activating mitochondrial uncoupling include extended lifespan, decreasing or suppressing harmful or unwanted cellular proliferation, increasing or restoring intestinal homeostasis and slowing or inhibiting tumor growth.

### BACKGROUND OF THE INVENTION

Nearly all animals exhibit behavior and physiologies that are tightly linked to a 24-hour circadian rhythm, controlled by endogenous circadian clocks. These clocks are composed of transcriptional feedback loops that regulate the expression of genes controlling diverse cellular functions. In Drosophila, the circadian transcriptional activators Clock and Cycle induce the oscillating expression of hundreds of target genes, including period (per) and timeless (tim), which encode repressors of Clock and Cycle activity (FIG. 1A). Because animals lose circadian rhythmicity as they age, it has long been hypothesized that loss of circadian regulation contributes to aging and limits lifespan. Specifically, there has been interest in the impact of circadian-regulated metabolism on lifespan and aging, as many mechanisms known to regulate organismal lifespan involve large metabolic changes, including many metabolism genes regulated by the circadian clock (Brown, 2016; Panda, 2016). Most reports investigating core molecular clock components and lifespan have examined the circadian transcriptional activators (i.e., Clock or Cycle) and found that disruption of these activators leads to detrimental metabolic function and shortened lifespan (Dubrovsky et al., 2010; Vaccaro et al., 2017). Studies involving loss of circadian transcriptional repressors (i.e., Period, Timeless, or the light receptor Cryptochrome) and the resulting influence on metabolism, healthspan, and lifespan have been more controversial (Ulgherait et al., 2016; Vaccaro et al., 2017; Wang et al., 2016a). While a number of studies have focused on the detrimental effects of loss of the transcriptional repressor mPer2 in the mouse (Lee et al., 2010), there is also evidence that loss of mPer2 in the whole organism, or in individual tissues, may have beneficial effects on specific aspects of healthspan and lifespan (Wang et al., 2016a; Wang et al., 2016b). For example, loss of mPer2 in the whole mouse has been associated with increased metabolic rate, lowered fat storage, increased leptin levels, and decreased insulin resistance compared to control animals, indicating the possibility of a favorable metabolic state under ad libitum feeding conditions (Grimaldi et al., 2010). The specific mechanisms underlying circadian-regulated metabolism and their roles in aging and longevity have up to now remained unclear.

### SUMMARY OF THE INVENTION

The present invention is based upon the surprising discovery that the activation of mitochondrial uncoupling has many beneficial effects including the delay of aging and extension of lifespan in otherwise healthy subjects. Activation of mitochondrial uncoupling also decreased or suppressed harmful cellular proliferation associated with aging and cancer. Activation of mitochondrial uncoupling also increased intestinal homeostasis and slowed tumor growth.

One embodiment of the present invention is a method of extending lifespan in a subject by administering a therapeutically effective amount of an agent which activates mitochondrial uncoupling. In some embodiments, the subject is healthy. In some embodiments, the subject is elderly. In some embodiments, the subject is suffering from cancer. In some embodiments, the subject is suffering from intestinal dysfunction or dysbiosis, or a lack of intestinal homeostasis. In some embodiments, the agent is administered to the intestinal tract.

A further embodiment of the present invention is a method of decreasing or suppressing harmful or undesirable cellular proliferation in a subject in need thereof by administering a therapeutically effective amount of an agent which activates mitochondrial uncoupling. In some embodiments, the cellular proliferation is associated with aging. In some embodiments, the cellular proliferation is associated with cancer. In some embodiments, the subject is suffering from another disease or disorder. In some embodiments, the cellular proliferation is stem cell proliferation. In some embodiments, the agent is administered to the intestinal tract.

A further embodiment of the present invention is a method of restoring or increasing intestinal homeostasis in a subject in need thereof by administering a therapeutically effective amount of an agent which activates mitochondrial uncoupling. In some embodiments, the intestinal disfunction is associated with aging. In some embodiments, the intestinal disfunction is associated with illness or injury, including but not limited to inflammatory bowel disease and, Crohn's disease. In some embodiments, the agent is administered to the intestines. Yet a further embodiment of the present invention is a method of slowing or inhibiting tumor growth in a subject in need thereof by administering a therapeutically effective amount of an agent which activates mitochondrial uncoupling. In some embodiments, the tumor is in an organ or tissue including but not limited to the: lungs; breast; liver; kidney; pancreas; gastrointestinal tract including esophagus, stomach, and small and large intestine; bones; and brain. In some embodiments, the administration of the agent which activates mitochondrial uncoupling is alone. In some embodiments, the administration can be in conjunction with other cancer therapeutics including but not limited to chemotherapeutic agents and immunomodulating agents.

Another embodiment of the present invention is a method of preventing or treating cancer in a subject in need thereof by administering a therapeutically effective amount of an agent which activates mitochondrial uncoupling. In some embodiments, the cancer is in an organ or tissue including but not limited to the: lungs; breast; liver; kidney; pancreas; gastrointestinal tract including esophagus, stomach, small and large intestine; bones; and brain. In some embodiments, the administration of the agent which activates mitochondrial uncoupling is alone. In some embodiments, the administration can be in conjunction with other cancer therapeutics including but not limited to chemotherapeutic agents and immunomodulatory agents.

As shown herein administration of a mild activator of mitochondrial uncoupling at low concentrations was enough to provide the beneficial effects and can be used in the methods of the present invention.

In some embodiments, the agent that is the activator of mitochondrial uncoupling is a small molecule. In some embodiments, the agent that is the activator of mitochondrial uncoupling is a chemical. In some embodiments, the agent that is the activator of mitochondrial uncoupling is a pharmaceutical.

In some embodiments, the agent is the agent is a nucleic acid which encodes a uncoupling protein (UCP), or an entire UCP gene, or a nucleic acid that is substantially homologous to a UCP gene, or a variant, mutant, fragment, homologue or derivative of a UCP gene.

The present invention also provides for kits comprising compositions and agents for practicing the invention.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIG. 1. Loss of the repressive arm of the transcriptional circadian clock extended lifespan independent of dietary protein restriction and reduced insulin-like signaling.

FIG. 1A is a schematic of core molecular clock components and circadian transcriptional feedback loop. FIG. 1B is a graph of percent survival relative to controls (dark line) of mutant males lacking cyc⁰¹ function (light line) showing reduced lifespan (p<0.0001 for each circadian mutant vs. control). FIG. 1C is a graph of percent survival relative to controls (dark line) of mutant males expressing dominant-negative clock (DaGS>UAS-DN-Clock) flies fed RU486 (light line) showing reduced lifespan (p<0.0001 for each circadian mutant vs. control). FIG. 1D is a graph of percent survival relative to controls (dark line) of tim⁰¹ mutants (light line) showing the mutants live longer than controls (p<0.001 for each circadian mutant vs. control). FIG. 1E is a graph of percent survival relative to controls (dark line) of per⁰¹ mutants (light line) showing the mutants live longer than controls (p<0.001 for each circadian mutant vs. control). FIG. 1F shows the percent survival of mutants rescued for Per expression in per⁰¹ null mutants using either UAS-per24 or UAS-per10 transgenes via ubiquitin-GAL4 as well as controls. FIG. 1G shows the percent survival of mutants rescued of Per expression in per⁰¹ null mutants using either UAS-per24 or UAS-per10 transgenes via timeless-GAL4. Both rescues in FIGS. 1F and 1G reverted per⁰¹ lifespan back to that of wild-type flies. FIG. 1H is a graph of the median lifespan of controls (dark line) and tim⁰¹ mutants (light line) showing the mutants live longer than controls on diets with different concentrations of yeast extract, except for the highest yeast diet (p<0.001 for each circadian mutant vs. control on each diet). FIG. 1I is a graph of the median lifespan of controls (dark line) and per⁰¹ mutants (light line) showing the mutants live longer than controls on diets with different concentrations of yeast extract, except for the highest yeast diet (p<0.001 for each circadian mutant vs. control on each diet). FIG. 1J shows western blot analyses of longevity-associated pathways performed at different circadian time points (n≥3 sample per time point/genotype/condition; 30 whole males/sample): phosphorylation of Akt at serine 505; of S6K at threonine 398; of AMPK at threonine 184; and lipidation of GFP-tagged Atg8a (unpaired two-tailed t-test). FIG. 1K is a graph of percent survival of per⁰¹ mutants with DaGS>UAS-DN-S6K (light line) and controls containing DaGS>UAS-DN-S6K (dark line) fed either RU486 (dashed lines) or vehicle (solid lines). FIG. 1L is a percent survival curve of per⁰¹ mutants (light line) and controls containing DaGS-GAL4 drivers and lacking UAS-transgenes (dark line) fed of RU486 (dashed lines) or vehicle (solid lines) during adulthood in did not influence lifespan. FIG. 1M is a graph of percent survival of per⁰¹ mutants (light line) and controls (dark line) containing either the Dilp2-GAL4 driver alone (solid lines) or Dilp2-GAL4>UAS-reaper and ablated for insulin-producing cells (dashed lines). n.s.=p>0.05,*=p<0.05,**=p<0.01,***=p<0.001; p-values were obtained by log-rank analysis (FIGS. 1F, 1G, 1L) and ANOVA followed by Tukey's post-hoc test (FIG. 1J).

FIG. 2. period mutants exhibited high metabolic rate due to mitochondrial uncoupling.

FIG. 2A is a graph showing per⁰¹ mutants (right hand bar) exhibited increased feeding rate (n>7 vials of 10 flies/condition, p<0.01) relative to controls (left hand bar). FIG. 2B is a graph of the decreased survival upon starvation of per⁰¹ mutants (light line) relative to controls (dark line) (n≥77 flies per condition, p<0.001). FIG. 2C is a graph of baseline levels of lipids (left) and rate of lipid utilization after 24 hours of starvation (right), as shown by quantification of triacylglyceride (TAG) levels in controls (left hand bars) and per⁰¹ mutants (right hand bars) (n=4 samples/condition, 5 flies/sample, both p<0.0001). FIG. 2D are representative images of thin-layer chromatography of TAG for quantification of triacylglyceride (TAG) levels in per⁰¹ mutants and controls (n≥4 samples/condition, 5 flies/sample) showing that per⁰¹ flies exhibited lower levels of lipids compared to control flies at baseline (0 hr) and increased rate of lipid utilization after 24 hours of starvation. FIG. 2E shows a graph of respiration rate (relative CO₂ production) in controls (left hand bars) and per⁰¹ mutants (right hand bars) fed either RU486 or vehicle (n=6 groups of 10 flies per condition). FIG. 2F is a graph of respiration rate (relative CO₂ production) over the circadian day in controls (bottom dark line) and per⁰¹ mutants (top light line). FIG. 2G is a graph of respiration rates (relative CO₂ production) of mutants (right hand bar) and controls (left hand bar) lacking UAS-transgenes fed RU486 or vehicle (n=6 groups of 10 flies per condition, ANOVA followed by Tukey's post-hoc test). FIG. 2H is a graph showing relative respiration rates (relative CO₂ production) in controls (left hand bars) and per⁰¹ mutants (right hand bars) after 24 hours of feeding with rotenone and oligomycin, 2,4 DNP, or stearic acid. FIG. 2I is a graph of oxygen consumption rate in controls (left hand bars) and per⁰¹ mutants (right hand bars) when stimulated through complex I, II, and IV relative to controls (n=4-6 oxygraph runs per condition). FIG. 2J are images of blue-native PAGE of purified mitochondria from mutants and controls (left) and graphs of quantitation of complex formation (right) showing that per⁰¹ and control lines had no difference in specific electron transport chain complex abundance (n=3 mitochondrial preps, p>0.05 for all comparisons). FIG. 2K are in-gel activity assay for complex I from mutants and controls (left) with quantification (right) that showed that per⁰¹ and control lines had no difference in complex I activity (n=3 mitochondrial preps, p>0.05 for all comparisons). FIG. 2L is a graph of leak respiration in controls and per⁰¹ mutants, using high-resolution respirometry on purified mitochondria (n=5 oxygraph runs per condition, p<0.001). FIG. 2M is a graph of membrane potential in controls (left hand bars) and per⁰¹ mutants (right hand bars), measured by JC-1 staining of purified mitochondria (n=6 mitochondrial preps per condition, p<0.001). FIG. 2N is a graph of percent recovery from cold shock in controls (dark line) and per⁰¹ mutants (light line) (n=32 flies per condition, p<0.001). n.s.=p>0.05, * =p<0.05, **=p<0.01, *** =p<0.001; p-values were obtained by unpaired two-tailed t-test (FIGS. 2A, 2C, 2F, 2I, 2J, 2K, 2L, 2M), ANOVA followed by Tukey's post-hoc test (FIGS. 2E, 2G, 2H), and log-rank analysis (FIGS. 2B, 2N).

FIG. 3. Endogenous uncoupling protein UCP4C was necessary for period mutant lifespan extension and sufficient to extend wild-type lifespan.

FIG. 3A is a graph of expression of Ucp4B, Ucp4C and Ucp4A in per⁰¹ mutants (right hand bars) relative to controls (left hand bars). FIG. 3B is a graph of the constitutive expression of Ucp4B in per⁰¹ mutants (light line) relative to controls (dark line). FIG. 3C is a graph of the constitutive expression of Ucp4C in per⁰¹ mutants (light line) relative to controls (dark line). FIG. 3D is a graph showing leak respiration by purified mitochondria in control flies with piggyback mutation of Ucp4B/C, per⁰¹ mutant flies with piggyback mutation of Ucp4B/C, control flies without piggyback mutation of Ucp4B/C, and per⁰¹ mutant flies without piggyback mutation of Ucp4B/C. FIG. 3E is a graph of mitochondrial membrane potential in control flies with piggyback mutation of Ucp4B/C, per⁰¹ mutant flies with piggyback mutation of Ucp4B/C, control flies without piggyback mutation of Ucp4B/C, and per⁰¹ mutant flies without piggyback mutation of Ucp4B/C. FIG. 3F is a graph showing the results of high-resolution respirometry on mitochondria purified from control flies with piggyback mutation of Ucp4B/C, per⁰¹ mutant flies with piggyback mutation of Ucp4B/C, control flies without piggyback mutation of Ucp4B/C, and per⁰¹ mutant flies without piggyback mutation of Ucp4B/C showing that per⁰¹ mutant mitochondria exhibited higher respiration rates under steady-state ATP production (n=6 mitochondrial preps per condition). FIG. 3G is a graph of percent recovery from cold shock in control flies with piggyback mutation of Ucp4B/C, per⁰¹ mutant flies with piggyback mutation of Ucp4B/C, control flies without piggyback mutation of Ucp4B/C, and per⁰¹ mutant flies without piggyback mutation of Ucp4B/C. FIG. 3H is a graph of percent survival in control flies with piggyback mutation of Ucp4B/C, per⁰¹ mutant flies with piggyback mutation of Ucp4B/C, control flies without piggyback mutation of Ucp4B/C, and per⁰¹ mutant flies without piggyback mutation of Ucp4B/C. FIG. 3I is a graph of percent survival of control flies (dark line) and per⁰¹ mutants (light line) with an induced knockdown of Ucp4A (dashed lines). FIG. 3J is a graph of percent survival of control flies (dark line) and per⁰¹ mutants (light line) with an induced knockdown of Ucp4B (dashed lines). FIG. 3K is a graph of percent survival of control flies (dark line) and per⁰¹ mutants (light line) with an induced knockdown of Ucp4C (dashed lines). FIG. 3L is a graph showing leak respiration by purified mitochondria in vehicle-fed DaGS>UAS- Ucp4C flies (left hand bar) and RU486-fed DaGS>UAS-Ucp4C flies (right hand bar) which induces constitutive UCP4C overexpression (light gray) (p<0.001). FIG. 3M is a graph of mitochondrial membrane potential in vehicle-fed DaGS>UAS- Ucp4C flies (left hand bars) and RU486-fed DaGS>UAS-Ucp4C flies (right hand bars) which induces constitutive UCP4C overexpression (light gray) (p<0.001). FIG. 3N is a graph of the relative CO₂ production of shows in vehicle-fed DaGS>UAS-Ucp4C flies (left hand bar) and RU486-fed DaGS>UAS-Ucp4C flies (right hand bar) which induces constitutive UCP4C overexpression (light gray) (p<0.001). FIG. 3O is a graph of cold shock recovery in vehicle-fed DaGS>UAS-Ucp4C flies (dark line) and RU486-fed DaGS>UAS- Ucp4C flies (light line) which induces constitutive UCP4C overexpression (p<0.001). FIG. 3P is a graph of percent survival in vehicle-fed DaGS>UAS-Ucp4C flies (dark line) and RU486-fed DaGS>UAS-Ucp4C flies (light line) which induces constitutive UCP4C overexpression (p<0.0001). FIG. 3Q is a graph of percent survival of flies with ubiquitous overexpression of UCP4C in otherwise wild-type flies was sufficient to extend lifespan (medium line) relative to driver-only controls (dark line) In per⁰¹ mutants (light line, solid), overexpression of UCP4C did not further extend the lifespan of per⁰¹ mutants (light line, dashed). n.s.=p>0.05, * =p<0.05, **=p<0.01, *** =p<0.001; p-values were obtained by unpaired two-tailed t-test (FIGS. 3A, 3L, 3M), ANOVA followed by Tukey's post-hoc test (FIGS. 3D, 3E), and log-rank analysis (FIGS. 3G, 3H, 3P, 3Q).

FIG. 4. Lifespan extension was mediated by loss of Per specifically in the intestine.

FIG. 4A is a graph showing percent survival of ubiquitous rescue of Per expression (dashed lines) in the per⁰¹ background (light line) and controls (dark line). Ubiquitous rescue of Per during adulthood was sufficient to revert per⁰¹ lifespan to control levels. FIG. 4B is a graph showing percent survival of neuronal rescue of Per expression (dashed lines) in the per⁰¹ background (light line) and controls (dark line). FIG. 4C is a graph showing percent survival of intestinal/gut rescue of Per expression (dashed lines) in the per⁰¹ background (light line) and controls (dark line). FIG. 4D is a graph showing percent survival of whole gut rescue of Per expression (dashed lines) in the per⁰¹ background (light line) and controls (dark line). FIG. 4E is a graph of percent survival of rescue of Per specifically (dashed lines) in intestinal stem cells (ISCs) and enteroblasts (EBs) during development or adulthood as compared to controls (dark lines) and per⁰¹ mutants (light lines). Rescue reverted perm lifespan to control levels. FIG. 4F is a graph of percent survival showing the loss of period through ubiquitous CRISPR-mediated deletion during adulthood extended lifespan of otherwise wild-type flies, with no further lifespan extension in per⁰¹ nulls. FIG. 4G is a graph of percent survival of tissue specific CRISPR-mediated deletion of period in the intestine of controls and per⁰¹ mutants. FIG. 4H is a graph of percent survival of tissue specific CRISPR-mediated deletion of period in the IScs and EBs of controls and per⁰¹ mutants. FIG. 4I is a graph of relative expression of period in the intestines in a adulthood-induced CRISPR deletion of period in ISC/EB populations by feeding of RU486 to flies containing 5961GS-GAL4>UAS-Cas9; per-gRNA reduced per transcript level in the intestine by more than 90% (n=3 samples 10 intestines/samples, p<0.001), while adulthood-induced CRISPR deletion of acp98AB in ISC/EB populations did not alter per transcript levels (n=3 samples 10 intestines/samples, p<0.934).p-values were obtained by logrank analysis.

FIG. 5. Upregulation of circadian-regulated UCP4C in the intestine was necessary for loss of Per-mediated lifespan extension and sufficient to extend wild-type lifespan.

FIG. 5A are representative images of the posterior midgut of control (left) and per⁰¹ mutants (right) stained with Hoechst (DNA) and mitochondrial membrane potential (TMRE) during the day and night (scale bar=30 μm). FIG. 5B is a graph quantifying the results of the images in FIG. 5A. While wild-type intestines (left hand) exhibited high membrane potential during the day and low membrane potential at night, per⁰¹ flies (right hand) exhibited low membrane potential at both times of day. FIG. 5C are representative images of control and per⁰¹ posterior midguts expressing cellular mitochondrial levels (mito-GFP) (scale bar=10 μm). FIG. 5D is quantification of mitochondrial fluorescence from controls (left hand) and per⁰¹ mutants (right hand) during day or night showed no significant changes in mitochondrial abundance (n=14 intestines per condition, ANOVA, p=0.2524). FIG. 5E is a graph of the percent survival of per⁰¹ mutants (light lines) with a knockdown of Ucp4C in the whole intestine (dashed light line). FIG. 5F is a graph of the percent survival of per⁰¹ mutants (light lines) with a knockdown of Ucp4C in the ISCs/EBs (dashed light line). Both knockdowns reverted per⁰¹ lifespan to control levels (dark lines). FIG. 5G is a graph of percent survival relative to controls (dark line), flies overexpressing Ucp4C (light line) in the neurons (p<0.0001 for each). FIG. 5H is a graph of percent survival relative to controls (dark line), flies overexpressing Ucp4C (light line) in the whole intestine (p<0.0001 for each). FIG. 5I is a graph of percent survival relative to controls (dark line), flies overexpressing Ucp4C (light line) in the ISCs and EBs (p<0.0001 for each). FIG. 5J is a graph of percent survival relative to controls (dark line), flies overexpressing Ucp4C (light line) in the ISCs and EBs (p<0.0001 for each). n.s.=p>0.05, * =p<0.05, **=p<0.01, *** =p<0.001; p-values were obtained by ANOVA followed by Tukey's post-hoc test (FIGS. 5B, 5D) and log-rank analysis (FIGS. 5E-5J).

FIG. 6. Loss of period preserved intestinal homeostasis via increased mitochondrial uncoupling and decreased ROS levels.

FIG. 6A are representative images of phospho-histone H3 at residue S10 (pHH3) staining of midguts from 45-day flies. FIG. 6B is a graph of the quantification of intestinal pHH3+staining in FIG. 6A showing that per⁰¹ mutants had lower levels of age-related hyperproliferation dependent on UCP4C expression and that overexpression of UCP4C caused a large reduction in mitotic cells (n=13-17 intestines, scale bar=40 μm). FIG. 6C is a graph of intestinal barrier dysfunction in smurf assays of 45-day old flies (from left to right-control, per⁰¹ mutants, control+upc4C IR, per⁰¹+ucp4c IR, ucp4c OE) showed per⁰¹ mutants had reduced populations with intestinal barrier dysfunction, a phenotype dependent upon Ucp4C expression, and that overexpression of Ucp4C also reduced intestinal barrier dysfunction. FIG. 6D are representative images for MitoSOX staining of aged posterior midguts in control esg-GAL4>GFP flies, per⁰¹; esg-GAL4>GFP, and esg-GAL4>GFP;UAS-Ucp4C flies (scale bars=35 μm (top) and 15 μm (bottom). FIG. 6E is a graph of GFP+cell count versus cell density for each fly type. FIG. 6F are graphs quantifying the images in FIG. 6D showing ROS output for the whole gut and ISC/EB populations. per⁰¹ mutants and Ucp4C-overexpressing flies exhibited delayed esg+cell overproliferation and lower ROS output of all posterior midgut cells, including ISC/EB populations. n.s.=p>0.05, * =p<0.05, **=p<0.01, *** =p<0.001; p-values were obtained by ANOVA followed by Tukey's post-hoc test (FIGS. 6B, 6C, 6E, 6F).

FIG. 7. Pharmacological reduction of ROS via uncoupling preserved intestinal homeostasis and extended lifespan.

FIG. 7A is a graph of percent survival relative to vehicle-fed control (dark line) of flies fed the mitochondrial uncoupler 2,4 DNP (light line) throughout their lifespan (p<0.0001 for each). FIG. 7B is a graph of percent survival relative to vehicle-fed control (dark line) of flies fed the mitochondrial uncoupler 2,4 DNP (light line) only during adulthood (p<0.0001 for each). FIG. 7C is the median lifespan versus dose of 2,4 DNP feeding, showing increased median lifespan in a dose-dependent manner FIG. 7D is a graph of the percent survival of flies fed the uncoupling compound butylated hydroxyl toluene (BHT) (light line) during adulthood versus vehicle-fed controls (dark line). The flies fed BHT had increased lifespan compared to vehicle-fed controls. FIG. 7E is a graph of food intake of flies fed either uncoupling compound 2,4 DNP or BHT relative to ethanol vehicle (n=8-10 groups of 10 flies, p=0.258). There was no significant difference between the three groups. FIG. 7F shows the median lifespan of control flies (dark line) and per⁰¹ mutants (light line) versus the concentration of DNP fed during adulthood. While feeding varying concentrations of 2,4 DNP during adulthood to control flies increased their median lifespan in a dose-dependent manner, no median lifespan increase was observed in per⁰¹ null mutants. FIG. 7G shows the median lifespan of control flies (dark line) and per⁰¹ mutants (light line) versus the concentration of BHT fed during adulthood. While feeding varying concentrations of BHT during adulthood to control flies increased their median lifespan in a dose-dependent manner, no median lifespan increase was observed in per⁰¹ null mutants. FIG. 7H are representative images of MitoSOX staining of posterior midguts in Esg-GAL4>GFP flies fed vehicle or DNP (scale bars=25 μm, top; 10 μm, bottom). FIG. 7I are graphs of the quantification of esg+cells, Mitosox staining of the entire posterior midgut, and Mitosox staining in ISCs/EBs marked by GFP in Esg-GAL4>GFP flies fed vehicle or DNP showing flies fed 2,4 DNP exhibited fewer esg+cells and thus lower ISC/EB overproliferation with age; decreased Mitosox staining of the entire posterior midgut and thus decreased mitochondrial superoxide output; and decreased MitoSOX staining specifically in ISCs/EBs marked by GFP. FIG. 7J are representative images of midguts from flies exhibiting loss of Notch-mediated tumor formation (scale bar=35 μm). FIG. 7K is a graph showing relative to vehicle-fed flies, flies with Notch-induced tumor formation that were fed 2,4 DNP exhibited delayed tumor formation as measured by GFP+area in the midgut (p<0.0001 for each). FIG. 7L is a graph of percent survival that shows relative to vehicle-fed flies (dark line), flies with Notch-induced tumor formation that were fed 2,4 DNP (light line) exhibited extended lifespans (p<0.0001 for each). n.s.=p>0.05, * =p<0.05, **=p<0.01, *** =p<0.001; p-values were obtained by log-rank analysis (FIGS. 7A, 7B, 7C, 7D, 7F, 7G, 7L), unpaired two-tailed t-test (FIG. 7I), and ANOVA followed by Tukey's post-hoc test (FIGS. 7E, 7K).

### DETAILED DESCRIPTION OF THE INVENTION

### Definitions

The term “subject” as used in this application means an organism that exhibits mitochondrial uncoupling such as protists, plants, fungi, insects, avians, reptiles, amphibians, and mammals Mammals include, but are not limited to, canines, felines, rodents, bovine, equines, porcines, ovines, and primates. Avians include, but are not limited to, parrots, fowls, songbirds, and raptors. Reptiles include, but are not limited to snakes, lizards, and tortoises. Amphibians include, but are not limited to, salamanders, newts, and frogs. Insects include, but are not limited to, bees, ants, termites, and mosquitos. Protists include, but are not limited to, giardia, trypanosomes, and plasmodium. Plants include, but are not limited to, trees, vegetables, grain, monocots, dicots, and legumes. Fungi include, but are not limited to, mushrooms, yeast, and mold. Thus, the invention can be used in veterinary medicine, e.g., to treat companion animals, farm animals, laboratory animals in zoological parks, and animals in the wild. The invention can also be used to aid other species, e.g., to treat crops, promote species survival, and control microorganisms. The invention is particularly desirable for human medical applications.

A therapeutically effective amount, or an effective amount, of a drug or agent is an amount effective to demonstrate a desired activity of the drug or agent. A “therapeutically effective amount” will vary depending on the agent, the disorder and its severity and the age, weight, physical condition and responsiveness of the subject to be treated.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “in need thereof” would be a subject known or suspected of having or being at risk of developing cancer. The term would also denote a subject with known or suspected or being at risk of developing a lack of intestinal homeostasis caused by age, disease or injury. The term would also denote a subject with known or suspected or being at risk of developing harmful or undesirable cellular proliferation. Lastly, the term would also denote an elderly or aged subject.

A subject in need of treatment would be one that has already been diagnosed with a disease or conditions. A subject in need of prevention would be one with a risk of developing a disease of condition.

The term “intestinal homeostasis” as used herein means the maintenance of the equilibrium between a subject and its intestinal microbiota. Intestinal homeostasis depends upon the mucosal barrier and permeability of the mucosal barrier. Lack of intestinal homeostasis is sometimes known as intestinal dysbiosis.

The term “agent” as used herein means a substance that produces or is capable of producing an effect and would include, but is not limited to, chemicals, pharmaceuticals, biologics, small organic molecules, antibodies, nucleic acids, peptides, and proteins.

As used herein, the term “small molecules” encompasses molecules other than proteins or nucleic acids without strict regard to size. Non-limiting examples of small molecules that may be used according to the methods and compositions of the present invention include, small organic molecules, peptide-like molecules, peptidomimetics, carbohydrates, lipids or other organic (carbon containing) or inorganic molecules.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “purified” and the like as used herein refers to material that has been isolated under conditions that reduce or eliminate unrelated materials, i.e., contaminants For example, a purified protein is preferably substantially free of other proteins or nucleic acids with which it is associated in a cell and a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include, but are not limited to, plasmids, phages, and viruses.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily be introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes and may be from the same or different organisms. A large number of vectors, including plasmid and fungal vectors, have been described for replication and/or expression in a variety of eukaryotic and prokaryotic hosts, and many appropriate host cells, are known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described herein.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates) and with charged linkages (e.g., phosphorothioates, phosphorodithioates). Polynucleotides may contain one or more additional covalently linked moieties, such as, for example, proteins (e.g., nucleases, toxins, antibodies, signal peptides, poly-L-lysine), intercalators (e.g., acridine, psoralen), chelators (e.g., metals, radioactive metals, iron, oxidative metals), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin. Sequence identity can be determined using any of a number of publicly available sequence comparison algorithms, such as BLAST, FASTA, DNA Strider, or GCG (Genetics Computer Group, Program Manual for the GCG Package, Version 7, Madison, Wis.).

The terms “substantially homologous” or “substantially similar” means when at least about 80%, and most preferably at least about 90 or 95%, 96%, 97%, 98%, or 99% of the nucleotides match over the defined length of the DNA sequences, as determined by sequence comparison algorithms, such as BLAST, FASTA, and DNA Strider. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols.1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

### Activation of Mitochondrial Uncoupling has Beneficial Effects

The present invention relates to methods of delaying aging, extending lifespan, increasing or restoring intestinal homeostasis, decreasing or suppressing harmful or undesirable cellular proliferation, slowing or inhibiting tumor growth, and preventing and treating cancer by activating mitochondrial uncoupling. The data shown herein support these beneficial effects of activating mitochondrial uncoupling and the administration of a mild pharmacological activator of mitochondrial uncoupling was shown to cause all of these beneficial effects.

Taken together, the data herein showed that the arrhythmic circadian mutant per⁰¹ which lack the regulator Period is long-lived, i.e., has delayed aging and extended lifespan, due to a non-canonical mechanism, additive with and independent of suppressed insulin signaling or TOR inhibition. Loss of Period caused high levels of mitochondrial uncoupling, resulting in hyperphagic, lean animals that maintain intestinal health with age (Example 3). Ablating circadian regulation via loss of Per or increasing circadian-regulated mitochondrial uncoupling specifically in the intestine was sufficient to lower intestinal reactive oxygen species (ROS) levels, preserving proliferative homeostasis and gut barrier function, and extending fly lifespan (Examples 4 and 5). Additionally, the application of a mild pharmacological activator of mitochondrial uncoupling in low doses caused significant lifespan extension (Examples 4, 8, and 9).

The results shown herein also revealed that uncoupling protein, UCP4C, overexpression in intestinal stem cells/enteroblasts (ISCs/EBs) alone was sufficient to lower ROS output of the entire intestine including enterocyte cells (ECs) (Examples 4 and 5). Arrhythmic circadian mutants lacking the regulator Period exhibited suppression of aging-associated intestinal stem cell proliferation. per mutants have uncoupled mitochondria and this was dependent on high expression of uncoupling protein. High uncoupling protein expression in intestinal stem cells was both necessary and sufficient for suppression of aging-associated intestinal stem cell proliferation (Example 6). The application of a mild pharmacological activator of mitochondrial uncoupling in low doses suppressed cellular proliferation and tumor growth (Examples 8 and 9).

Alterations in mitochondrial metabolism and oxidative state are conserved in the process of mammalian intestinal stem cell differentiation (Berger et al., 2016; Myant et al., 2013). Thus, this work supports a role for oxidative stress in the maintenance of intestinal stem cell identity with age, a role that is likely to be evolutionarily conserved. Together with inhibition of tumor growth, the data also suggested that mitochondrial uncoupling induces a metabolic state that inhibits cellular proliferation.

The data presented herein provide insight into circadian-regulated mitochondrial function, aimed at improving intestinal healthspan and organismal longevity, and implicates stimulation of mitochondrial uncoupling as a potential therapeutic for control of stem cell overproliferation and delay of aging as well as cancer and disease related to lack of intestinal homeostasis.

There are five families of uncoupling proteins in mammals UCP1-5. UCP4 and UCP 5 are known as SCL25A27 and SLC25A14. UCP1 is only detected in brown adipocytes, UCP2 is present in many organs and cell types, and UCP3 is predominantly expressed in skeletal muscle. UCP2 and UCP3 limit the level of reactive oxygen species (ROS). Drosophila only have homologs of UCP4 and UCP5. Moreover, Drosophila UCP4 is now thought to be an ancient progenitor to the other UCPs.

Pharmacological agents that activate mitochondrial uncoupling have previously been discovered and marketed as diet pills. Mitochondrial uncoupling is the metabolism that occurs in brown fat.

### Methods and Compositions for Activating Mitochondrial Uncoupling

As shown herein, activation of mitochondrial uncoupling can be beneficial. Mitochondrial uncoupling extended lifespan in healthy subjects (Examples 4, 6, and 8) and those with cancer and intestinal disfunction, i.e., lack of intestinal homeostasis (Examples 7 and 9). Mitochondrial uncoupling also decreased and suppressed the overproliferation of cells related to both aging (Examples 6 and 7) and cancer (Example 9). Mitochondrial uncoupling also slowed tumor growth (Example 9). Mitochondrial uncoupling also improved and promoted intestinal health and restores intestinal homeostasis which in turn increases lifespan (Example 7). Elderly in particular suffer from intestinal disfunction including a lack of intestinal homeostasis. However, lack of intestinal homeostasis is related to other diseases and conditions including but not limited to inflammatory bowel disease and Crohn's disease as well as other autoimmune diseases such as type 1 diabetes.

Mitochondrial uncouplers are agents that decrease the efficiency of ATP production. Several agents are known to activate mitochondrial uncoupling including many small molecules. Any agent that is an activator of mitochondrial uncoupling can be used in the methods of the present invention. Exemplified herein are the use of mifepristone (RU486) (which induces the constitutive overexpression of UCPs), stearic acid, 2,4-dinitrophenol (2,4 DNP or DNP) and butylated hydroxytoluene (BHT). Administration of RU486 increased lifespan (Example 3). Also shown herein was that administration of low concentrations of the latter two agents were sufficient to decrease or suppress cellular proliferation, slow or inhibit tumor growth, and extend lifespan (Examples 8 and 9).

Agents which activate mitochondrial uncoupling include lipophilic weak acids. The majority of compounds possessing protonophoric activity are lipophilic weak acids with a pKa in the range of 5-7. The general structural requirements for uncoupling activity include the presence of an acid-dissociable group, bulky lipophilic groups, and a strong electron withdrawing moiety. The most representative agents of this class are substituted phenols, trifluoromethylbenzimidazoles, salicylanilides, and carbonyl cyanide phenylhydrazones.

DNP is an example of a substituted phenol. Other substituted phenols include dicoumarol and 2,6-di-tert-butyl-4-(2′,2′-dicyanovinyl)phenol. Important structures of substituted phenols for activating mitochondrial uncoupling are hydrophobicity, acidity, and the stability of ionized intermediate in the lipid phase of a membrane.

Salicylanilide derivates possess bacteriostatic and fungicidal activity and members of this class are widely used as anticestodal, antitrematodal, and antihelmintic drugs. Many of these chemicals are protonophoric uncouplers of mitochondrial oxidative phosphorylation. Structure-activity relationships with salicylanilide derivatives show that that both hydrophobicity and electron-withdrawing power were necessary for uncoupling activity.

Niclosamide is an example of an oral salicylanilide derivative approved by the US Food and Drug Administration (FDA) since 1960 for human use in the treatment of intestinal tapeworm infections. Niclosamide is a hydrogen ionophore that translocates protons across the mitochondrial membrane resulting in mitochondrial uncoupling and futile cycles of glucose and fatty acid oxidation. It has an excellent safety profile in human as transient mild mitochondrial uncoupling is tolerable in normal cells. It is currently available in a 500 mg chewable tablet.

A further example of a salicylanilide derivative is nitazoxanide an anti-helminth drug. Nitazoxanide is currently available in two oral dosage forms: a tablet (500 mg) and an oral suspension (100 mg per 5 ml when reconstituted). An extended release tablet (675 mg) has been used in clinical trials for chronic hepatitis C

Carbonyl cyanide p-trifluromethoxyphenyl-hydrazone (FCCP) and carbonyl cyanide meta-chlorophenylhydrazone are examples of carbonyl cyanide phenylhydrazones.The uncoupling activity of these phenylhydrazones correlates well with their protonophoric activity.

Still a further example of a lipophilic weak acid novel mitochondrial protonophore uncoupler is (2-fluorophenyl) {6-[(2-fluorophenyl)amino](1,2,5-oxadiazolo[3,4-e]pyrazin-5-yl)}amine, named BAM15 (Kenwood et al. 2013).

Other types of agents which activate uncoupling include endogenous and exogenous fatty acids and fatty acid—like compounds, such as perfluorodecanoic acid, sulfuramide, and methyl-substituted hexadecanedioic acid. In particular, long-chain fatty acids have been shown to activate UPC1 and UPC2 transcription.

Membrane-active peptides can also be used to activate mitochondrial uncoupling. These membrane-active peptides can form channels, selective to alkaline cations and/or protons, or they can form large pores allowing permeation of high-molecular-weight solutes. These peptides possess a high affinity toward mitochondrial membranes because insertion of the peptide into the membrane and/or pore formation is driven by the electrical potential. These peptides induce nonspecific permeability changes, which results in oncotic swelling and disruption of the charged organelles.

Examples of a pore-forming peptides are alamithicin and mastoparan. Mastoparan, an amphipathic peptide from wasp bee venom, induces the opening of a Ca²+-dependent cyclosporine A—sensitive mitochondrial permeability transition pore. At higher concentrations, mastoparan depolarizes the mitochondrial inner membrane by acting on the lipid phase with no apparent involvement of the permeability transition pore. Other short amphipathic peptides (especially of fungal origin) are known to uncouple oxidative phosphorylation in mitochondria at submicromolar concentrations by rendering the inner mitochondrial membrane permeable to various solutes.

The effect of the peptides on mitochondrial integrity has been shown to be dependent on concentration. At low peptide/ mitochondria ratios, signaling peptides induce a gradual lysis of the outer membrane and a release of enzymes from the intermembrane space. At higher peptide/ mitochondria ratios, the permeability of the inner membrane increases, leading to complete uncoupling of respiration and dissipation of the membrane potential.

Signal peptide—induced uncoupling is of great interest and importance for several reasons. These peptides are synthesized inside the cell and they are naturally and selectively targeted to the mitochondria by their very structure. In the case of malfunctioning of the protein import machinery, such peptides may accumulate within mitochondria, discharging the membrane potential and eliminating damaged organelles. Recently, signaling peptides have been explored as prototypes for creating new drugs selectively targeted to mitochondria within the cell.

Retinoic acid and carotenoids have been shown to increase UCP1.

See generally Wallace and Starkov 2000.

The methods of the present invention can also be achieved by administering an effective amount of a nucleic acid encoding one or more uncoupling proteins, UCP1-5 and include nucleic acids which encodes a UCP protein, or an entire UCP gene, or a nucleic acid that is substantially homologous to a UCP gene, or a variant, mutant, fragment, homologue or derivative of an UCP gene that produces a protein that maintains or increases its function.

In an embodiment, the variant of the nucleic acid encoding the UCP has at least 81% sequence identity with the sequence of the nucleotide of which it is a variant. Thus, preferably, the variant of the nucleotide has at least 81%, 82%, 83%, 84%, 85%, 86%, 87%, 88%, 89%, 90%, 91%, 92%, 93%, 94%, 95%, 96%, 97%, 98% or 99% sequence identity with the sequence of the Ube3A nucleotide.

DNA or other nucleic acids such as mRNA can also be used in the method.

The sequences for the various UCP genes can be found on the National Center for Biotechnology Database and can be used to manufacture variants, mutants, fragments, homologues and derivatives which maintain or have increased function.

UCP2 is encoded by the UCP2 gene on chromosome 11 (Gene ID: 7351). UCP4 is encoded by the SLC25A27 gene on chromosome 6 (Gene ID: 9481). UCPS is encoded by the SLC25A14 gene on the X chromosome (Gene ID: 9016).

The nucleic acid molecule encoding the UCP family member protein (e.g., UCP4) may comprise a viral vector, e.g., a recombinant adeno-associated virus (AAV) vector, encoding the UCP family member protein (e.g., UCP4).

Exemplary viral vectors include, but are not limited to, recombinant retroviruses, alphavirus-based vectors, oncoretroviral vectors, adenovirus vectors, Herpes simplex virus vectors, lentiviruses and adeno-associated virus (AAV) vectors.

The vectors comprising the present nucleic acid may be delivered into host cells by a suitable method. Methods of delivering the present composition to cells may include transfection of nucleic acids or polynucleotides (e.g., using reagents such as liposomes or nanoparticles); electroporation, delivery of protein, e.g., by mechanical deformation (see, e.g., Sharei et al. Proc. Natl. Acad. Sci. USA (2013) 110(6): 2082-2087); or viral transduction.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Moreover, inducible and tissue specific expression of an RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others.

In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

Vectors according to the present disclosure can be transformed, transfected or otherwise introduced into a wide variety of host cells. Numerous methods of transfection are known to the ordinarily skilled artisan, for example, lipofectamine, calcium phosphate co-precipitation, electroporation, DEAE-dextran treatment, microinjection, viral transduction, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

### Pharmaceutical Compositions and Methods of Administration

The present invention encompasses the administration of an activator of mitochondrial uncoupling, which in some embodiments a small molecule or a peptide. In some embodiments, the activator is a nucleic acid molecule encoding a UCP family member protein. In some embodiments, the nucleic acid further comprises a vector. The present invention includes pharmaceutical compositions comprising the various activators of mitochondrial uncoupling and the administration of such.

Methods of administration include oral; mucosal, such as nasal, sublingual, vaginal, buccal, or rectal; parenteral, such as subcutaneous, intravenous, bolus injection, intramuscular, or intra-arterial; or transdermal administration to a subject. Thus, the activator of mitochondrial uncoupling must be in the appropriate form for administration of choice. It has been shown herein that the activation of mitochondrial uncoupling in the intestine is sufficient to extend lifespan, decrease or suppress cellular proliferation as well as restoring intestinal health and homeostasis. Thus, preferred modes of administration would include those that deliver the agent to the intestines, including but not limited to oral and rectal administration.

Such compositions for administration may comprise a therapeutically effective amount of the agent and a pharmaceutically acceptable carrier. The phrase “pharmaceutically acceptable” refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans. “Carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

These compositions can take the form of solutions, suspensions, emulsions, tablets, pills, capsules, powders, sustained-release formulations, cachets, troches, lozenges, dispersions, suppositories, ointments, cataplasms (poultices), pastes, powders, dressings, creams, plasters, patches, aerosols, gels, liquid dosage forms suitable for parenteral administration to a patient, and sterile solids (e.g., crystalline or amorphous solids) that can be reconstituted to provide liquid dosage forms suitable for parenteral administration to a patient. Such compositions will contain a therapeutically effective amount of the agent, preferably in purified form, together with a suitable form of carrier so as to provide the form for proper administration to the patient. The formulation should suit the mode of administration.

Pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

Pharmaceutical compositions adapted for rectal administration may be provided as suppositories or enemas.

Pharmaceutical compositions adapted for transdermal administration may be provided as discrete patches intended to remain in intimate contact with the epidermis of the recipient over a prolonged period of time.

Pharmaceutical compositions adapted for nasal and pulmonary administration may comprise solid carriers such as powders which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

Pharmaceutical compositions adapted for vaginal administration may be provided as pessaries, tampons, creams, gels, pastes, foams or spray formulations.

Pharmaceutical compositions adapted for parenteral administration include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerine, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Delivery vehicles such as liposomes, nanocapsules, microparticles, microspheres, lipid particles, vesicles, and the like can be used to deliver the agent which activates the mitochondrial uncoupling directly to specific tissue including intestinal and various tumor tissue. The present composition may be administered in a local or systemic manner, for example, via injection directly into the desired target site, e.g., in a depot or sustained release formulation. The composition may be administered in a targeted drug delivery system, for example, in liposomes or nanoparticles coated with tissue-specific or cell-specific ligands/antibodies. The liposomes or nanoparticles will be targeted to and taken up selectively by the desired tissue or cells, e.g., tumor cells.

The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868; and 5,795,587).

Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500Å, containing an aqueous solution in the core.

Alternatively, nanocapsule or nanoparticle formulations may be used. Nanocapsules can generally entrap substances in a stable and reproducible way. NPs are colloidal carriers that can have a natural or synthetic origin and can vary from 1 to 1000 nm in size. Synthetic NPs may be prepared from polymeric materials such as poly(ethylenimine) (PEI), poly(alkylcyanoacrylates), poly(amidoamine) dendrimers (PAMAM), poly(ϵ-caprolactone) (PCL), poly(lactic-co-glycolic acid) (PLGA), polyesters (poly(lactic acid) (PLA), or from inorganic materials such as gold, silicon dioxide (silica), among others. These carriers can transport drugs by adsorbing, entrapping or bounding covalently to them. Natural NPs are produced from natural polymers, such as polysaccharides (chitosan and alginate), amino acids (poly(lysine), poly(aspartic acid) (PASA)), or proteins (gelatin and albumin). Natural NPs have the advantage of providing biological signals to interact with specific receptors/transporters expressed by endothelial cells.

Optionally, the compositions of the invention may contain, other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

Selection of a therapeutically effective amount or dose will be determined by the skilled artisan considering several factors which will be known to one of ordinary skill in the art. Such factors include the particular form of the inhibitor, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

The agents which activate mitochondrial uncoupling can be administered in conjunction with other therapeutic agents, including but not limited to inhibitors of cell proliferation, chemotherapeutic agents and immunomodulating agents as well as other therapeutic agents for a particular disease or condition, such as Crohn's disease.

Subjects Benefitting from Mitochondrial Uncoupling

It has been discovered that the activation of mitochondrial uncoupling extends lifespan in both healthy subjects and those with cancer and lack of intestinal homeostasis, inhibits or slows tumor growth, decreases or suppresses harmful or unwanted cellular proliferation, and improves and promotes intestinal health by increasing or restoring intestinal homeostasis.

Since activation of mitochondrial uncoupling extends lifespan, all subjects including those who are healthy, would benefit from the administration of an which activates mitochondrial uncoupling.

Subjects who would benefit from administration of an agent which activates mitochondrial uncoupling would be those with cancer including but not limited to tumors in the: lungs; breast; liver; kidney; pancreas; gastrointestinal tract including esophagus, stomach, and small and large intestine; bones; and brain. Administration of the agent which activates mitochondrial uncoupling could be alone or in conjunction with other cancer therapeutics including but not limited to inhibitors of cell proliferation, chemotherapeutic agents and immunomodulating agents as well as CRISPR.

Additional subjects who have intestinal disfunction, including lack of intestinal homeostasis, would benefit from the administration of an agent which activates mitochondrial uncoupling as it has been shown herein that this treatment improves intestinal homeostasis and health, thus in turn extending lifespan which is often related to mortality. Subjects with lack of intestinal homeostasis include the elderly as well as those who have had a loss of intestinal homeostasis from illness or injury.

Additionally, subjects who have or at risk of harmful or undesirable cellular proliferation from aging or cancer would also benefit from the administration of an agent which activates mitochondrial uncoupling.

its

The present invention also provides kits comprising the components of the combinations of the invention in kit form. A kit of the present invention includes one or more components including, but not limited to, an agent which activates mitochondrial uncoupling., as discussed herein, in association with one or more additional components including, but not limited to a pharmaceutically acceptable carrier. The therapeutic agent can be formulated as a pure composition or in combination with a pharmaceutically acceptable carrier, in a pharmaceutical composition.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, and proper storage conditions,

### EXAMPLES

The present invention may be better understood by reference to the following non-limiting examples, which are presented in order to more fully illustrate the preferred embodiments of the invention. They should in no way be construed to limit the broad scope of the invention.

### Example 1

### Materials and Methods

### Fly Srains

Cycle (cyc⁰¹) period (per⁰¹) and timeless (tim¹⁰) mutants outcrossed to the Canton-S (CS) background as controls were obtained from Jaga Giebultowicz. pBAC-Ucp4c^{e03988}, UAS-DN-S6K (6911) were obtained from the Bloomington Stock Center. UAS-Ucp4a-RNAi, UAS-Ucp4b-RNAi, UAS-Ucp4c-RNAi were obtained from the Vienna Drosophila Resource Center. UAS-Ucp4c was received from flyORF. UAS-GFP-Atg8 from Eric Baehrecke, Ubi-GAL4 from Marc Dionne, Esg-GAL4, NP3084-GAL4, Daughterless-Gene-Switch (DaGS), Elav-Gene-Switch (ELAV-GS) and TIGS-2 (TIGS-GAL4), 5961GS-GAL4, DILP2-GAL4 and UAS-Reaper from David W. Walker, UAS-per10 and UAS-per24, and UAS-DN-Clock from Amita Sehgal, Esg^{ts}-GAL4, and UAS-Notch-RNAi were from Ben Ohlstein. All experiments with multiple transgenes used flies that have undergone 12 generations of out-crossing into a w¹¹¹⁸ Canton-S (CS) control and/or per⁰¹, w¹¹¹⁸ Canton-S (CS) mutant background.

### UAS-CRISPR Line Construction

Multiple gRNAs targeting per, or acp98AB were constructed according to (Port and Bullock, 2016). In short, pCFD6 (Addgene #73915) was digested with Bbsl-HF (NEB #R3539S) and the linearized plasmid gel purified. For each construct, inserts were generated in three separate PCR reactions using pCFD6 as the template and the primers listed below. The resulting three inserts and the pCFD6 backbone were then assembled by NEBuilder HiFi DNA Assembly (NEB #E2621L) for each construct. Each construct was then micro-injected at integration site Su(Hw)attP5 (Bestgene Inc.) and Sanger sequenced.

UAS-per-gRNA, guide RNA targets:
(SEQ ID NO: 1)
1. GCTTTTCTACACACACCCGG
(SEQ ID NO: 2)
2. CACGTGCGATATGATCCCGG
(SEQ ID NO: 3)
3. GGAGTCCACACACAACACCA
(SEQ ID NO: 4)
4. TACTCGTCCATAGACCACGC
(SEQ ID NO: 5)
per_PCR1fwd-GCGGCCCGGGTTCGATTCCCGGCCGATGCAGCTTTTCTA
CACACACCCGGGTTTTAGAGCTAGAAATAGCAAG
(SEQ ID NO: 6)
per_PCR1rev-CCGGGATCATATCGCACGTGTGCACCAGCCGGGAATCGA
ACCC
(SEQ ID NO: 7)
per_PCR2fwd-CACGTGCGATATGATCCCGGGTTTTAGAGCTAGAAATAG
CAAG
(SEQ ID NO: 8)
per_PCR2rev-TGGTGTTGTGTGTGGACTCCTGCACCAGCCGGGAATCGA
ACCC
(SEQ ID NO: 9)
per_PCR3fwd-GGAGTCCACACACAACACCAGTTTTAGAGCTAGAAATAG
CAAG
(SEQ ID NO: 10)
per_PCR3rev-
ATTTTAACTTGCTATTTCTAGCTCTAAAACGCGTGGTCTATGGACGAGT
ATGCACCAGCCGGGAATCGAACCC
UAS-Ctrl-gRNA, guide RNA targets:
(SEQ ID NO: 11)
1. GTGTCCCCTTATTCGTGCGG
(SEQ ID NO: 12)
2. CACACTATCAAAGGATGACG
(SEQ ID NO: 13)
3. ATAAGGGGACACACTATCAA
(SEQ ID NO: 14)
4. AGTGTGTCCCCTTATTCGTG
(SEQ ID NO: 15)
acp98AB_PCR1fwd-GCGGCCCGGGTTCGATTCCCGGCCGATGCAGTGTC
CCCTTATTCGTGCGGGTTTTAGAGCTAGAAATAGCAAG
(SEQ ID NO: 16)
acp98AB_PCR1rev-CGTCATCCTTTGATAGTGTGTGCACCAGCCGGGAA
TCGAACCC
(SEQ ID NO: 17)
acp98AB_PCR2fwd-
CACACTATCAAAGGATGACGGTTTTAGAGCTAGAAATAGCAAG
(SEQ ID NO: 18)
acp98AB_PCR2rev-TTGATAGTGTGTCCCCTTATTGCACCAGCCGGGAA
TCGAACCC
(SEQ ID NO: 19)
acp98AB_PCR3fwd-
ATAAGGGGACACACTATCAAGTTTTAGAGCTAGAAATAGCAAG
(SEQ ID NO: 20)
acp98AB_PCR3rev-
ATTTTAACTTGCTATTTCTAGCTCTAAAACCACGAATAAGGGGA
CACACTTGCACCAGCCGGGAATCGAACCC

### Fly Media

Drosophila were reared from embryos in low-density bottles with standard medium containing 3.8% glucose, 1.9% sucrose, 3% yeast nutritional flake (Lab Scientific), 6.5% cornmeal, 0.8% agar, supplemented with 1.5% methylparaben mix (10% methylparaben in ethanol) and 1% propionic acid. Adult flies that eclosed within a 24 hour period were collected and transferred to “adult medium” containing 3.8% glucose, 1.9% sucrose, 8% cornmeal, 1% agar, and either 0.01%, 0.5%, 3%, 5%, or 10% yeast extract (Difco) supplemented with 1.5% methylparaben mix and 1% propionic acid for lifespan and biochemical analysis. All percentages given in wt/v except methylparaben mix, propionic acid given in v/v.

### Drug Supplementation in Media

All drugs were supplemented into cooled (65° C.) liquid adult medium (containing 3% yeast extract) following preparation to the following final concentration(s): RU486 100μg/mL. RU486 (Cayman Chemical) dissolved in ethanol was supplemented into medium after eclosion at a final concentration of 100 μg/mL (with the exception of DaGS>UAS-Ucp4c and controls, 5 μg/mL) vehicle controls were supplemented with same volume of ethanol alone. Developmental induction was achieved by adding RU486 at concentration of 5 μg/mL, or ethanol vehicle, followed by adults being aged on standard food containing no RU486. For uncoupler feeding 2,4 DNP or BHT purchased from Sigma was dissolved in ethanol fresh at the time of making food and added in to the media, at the indicated concentrations. 2,4 DNP (0.01-0.1 mM) and BHT (0.1-1 mM).

### Lifespan Analysis and Starvation

Newly eclosed flies (˜24 hr) were collected and allowed to mate for 48 hr. Female and male flies were separated and maintained at a density of 30-35 flies per vial in a humidified, temperature-controlled (25° C.) incubator with a 12-hour light-dark cycle. For starvation analysis, flies were aged for 7 days in vials containing indicated diets and then transferred to 1% agar in a humidified, temperature-controlled incubator with 12-hour light-dark cycle at 25° C. Death was scored every 1-2 days, with live flies transferred to fresh vials every 2-3 days. Statistical significance was determined by log-rank analysis.

### Western Blot Analysis

Whole-body lysates of 10-day-old male flies (30 flies/sample/timepoint) were separated by SDS-PAGE using standard procedures. Membranes were probed with antibodies against AMPK phospho-T184 at 1:1000 (Cell Signaling, 40H9); anti-phospho-S6K T398 (Cell Signaling, 9209), anti-GFP (Cell Signaling), anti-phospho AKT s505, and pan AKT at 1:1000 (Cell Signaling); and horseradish peroxidase (HRP)-conjugated monoclonal mouse anti-actin antibody at 1:5000 (Sigma-Aldrich). Rabbit antibodies were detected using HRP-conjugated anti-rabbit IgG antibodies at 1:2000 (Cell Signaling). Mouse antibodies were detected using HRP-conjugated anti-mouse IgG antibodies 1:2000 dilution (Cell Signaling). ECL chemiluminescence reagent (Pierce) was used to visualize horseradish peroxidase activity and detected by CCD camera using a Kodak Image Station. A minimum of four independent samples of each condition were used for statistical analysis and quantification.

### Quantitative Real-Time PCR

30 whole male flies, or 10 whole intestines per biological replicate were used for total RNA extraction via TRIzol reagent (Invitrogen) following manufacturer protocols. Samples were treated with DNase, and then cDNA was synthesized by Revertaid First Strand cDNA Synthesis Kit (Thermo Scientific). A minimum of four independent samples were used for statistical analysis and quantification. Equalized amplicons of Actin5C were used as a reference gene to normalize expression via the following primer sets:

(SEQ ID NO: 21)
Ucp4A-fwd- TTTGACTACGCGGACTCATTC
(SEQ ID NO: 22)
Ucp4A-rev- CGCGGTATTGCATATTGGACTT
(SEQ ID NO: 23)
Ucp4B-fwd- AACACAGTCTTTAGGCCAGCA
(SEQ ID NO: 24)
Ucp4B-rev- CCGTGAGGTAGAGTTCAACCG
(SEQ ID NO: 25)
Ucp4C-fwd- ACAAACGTCGCTGATCCACTA
(SEQ ID NO: 26)
Ucp4C-rev- GGAAGACACACGACTCGGC
(SEQ ID NO: 27)
period-fwd- GGTTGCTACGTCCTTCTGGA
(SEQ ID NO: 28)
period-rev- TGTGCCTCCTCCGATATCTT
(SEQ ID NO: 29)
Actin5C-fwd- TTGTCTGGGCAAGAGGATCAG
(SEQ ID NO: 30)
Actin5C-rev- ACCACTCGCACTTGCACTTTC

### Cold Shock Recovery Assay

7-10 day old males were anesthetized on ice and individually placed in vials and loaded into a DAMS activity monitor. Flies were maintained at 4° C. for 1 hour, then placed in a 25° C. incubator and allowed to recover for 4 hr. Individual flies were scored as recovered at the time 3 beam breaks were recorded by the activity monitor system. A minimum of 22 flies per condition were used for statistical analysis (log-rank analysis).

### Quantification of Triglycerides Levels

Lipids were extracted from five whole flies in a chloroform:ethanol solution (2:1 vol/vol), and nonpolar lipids (fatty acid, triacylglycerol) were separated by thin-layer chromatography with an n-hexane/diethylether/glacial acetic acid solution (70:30:1, vol/vol/vol). Plates were air-dried and stained (with 0.2% Amido Black 10B in 1 M NaCl), and lipid bands were quantified by photo densitometry using ImageJ software. Densitometry values were normalized to the mass of the homogenized flies used in the sample following a reference standard of pre-measured coconut oil or butter. A minimum of six independent samples were used for statistical analysis and quantification.

### Intestinal Barrier Dysfunction Assay

The “smurf fly”/intestinal barrier dysfunction assay was performed similarly to Rera et al. 2012. Flies were aged on regular medium until the day of the smurf assay. Dyed medium was prepared by the addition of FD&C Blue No. 1 at a final concentration of 2.5% wt/vol. A fly was counted as a smurf when dye coloration was observed outside the digestive tract. A minimum of 45 flies were used for each condition. Comparisons of smurf proportion per time point were carried out using binomial tests to calculate the probability of having as many smurfs in population A as in population B.

### Feeding Assay

Analysis of capillary feeding (“the CAFE assay”) was performed similarly to Ja et al.

2007 with minor modifications. Briefly, 10 flies were placed in vials with wet tissue paper as a water source and a capillary food source (3.8% glucose, 1.9% sucrose, 3% yeast extract, and 0.2% FD&C Blue No. 1). Feeding was monitored for at least 24 hr, replacing depleted capillaries as necessary. A minimum of 8 groups of 10 flies per condition were used for each experiment.

### CO₂ Respiration Assay

Measurement of CO₂ production was performed similarly to Yatsenko et al. 2014. Briefly, 10 flies were placed in a sealed pipette tip-capillary respirometer (without anesthesia) containing a small amount of soda-lime. Respirometers were placed capillary side down in dH20 containing 0.1% blue food dye in a sealed TLC chamber placed in a 25° C. incubator. Empty sealed respirometers (containing no flies) were used to subtract blue dye background migration from experimental (fly-containing) samples. Flies were tested for 2 hr between at circadian timepoints, and CO₂ production rate was indirectly determined by the amount of liquid movement up the capillary per hour after background subtraction. At least 6 groups of 10 flies were used per genotype for quantification for each condition. For mitochondrial drug feeding experiments, flies were placed on food containing the following compounds for 24 hr: 500 μM Oligomycin, 1 mM rotenone, 5 mM 2,4 DNP, 0.5% stearate, or ethanol vehicle controls.

### Mitochondrial Purification for Downstream Assays

50 whole flies were gently crushed in chilled mitochondrial isolation medium (MIM) (250 mM sucrose, 10 mM Tris-HCl (pH 7.4), 0.15 mM MgCl₂) passed through a 40 μm filter basket and spun twice at 1000 ×g for 5 min at 4° C. to remove nuclei and debris. The supernatant was then spun at 4000×g, for 10 min at 4° C. The pellet, containing the mitochondria, was washed once in fresh MIM buffer and then total protein content of the resulting pellet was determined by Bradford Assay.

### Mitochondrial Oxygen Consumption Measurements

Protein content of purified mitochondria was normalized, and samples were resuspended in modified MiR05 buffer (0.5 mM EGTA, 3 mM MgCl₂, 60 mM K-lactobionate, 20 mM taurine, 10 mM KH₂PO₄, 20 mM HEPES, 110 mM sucrose, and 0.25 g/L BSA, pH 7.2). Mitochondrial respiration was measured at 25° C. using high-resolution respirometry (Oxygraph-2 k, Oroboros). To determine max ADP stimulated respiration initiated through complex I, and II the following sequential injections were made (final concentration in chamber): 5 mM glutamate plus 5 mM malate, 5 mM succinate, 1 mM ADP. To determine non-phosphorylating leak respiration, 3 μM oligomycin was then added. To measure reserve electron transport capacity of the mitochondria, carbonyl cyanide m-chlorophenyl hydrazine (CCCP) 0.5 mM stock was titrated in to the chamber at 1 μL increments. Residual non-mitochondrial oxygen consumption was determined by the addition of 1 μM rotenone, and 2.5 μM antimycin A. Respiration rates of individual complexes I, II, and IV were determined by the following injection/reaction schema (final concentrations in chamber): 5 mM glutamate plus 5 mM malate, 1 mM ADP, 0.5 μM rotenone, 5 mM succinate, 2.5 μM antimycin A, and 1 mM ascorbate plus 0.25 mM N′-tetramethyl-1,4-phenylenediamine, 0.5 mM KCN. A minimum of four independent mitochondrial preps were run for each condition.

### Membrane Potential JC-1 Assay

Protein content of purified mitochondria was normalized, and samples were resuspended at a concentration of 200 μg/mL in modified MiR05 buffer (0.5 mM EGTA, 3 mM MgCl₂, 60 mM K-lactobionate, 20 mM taurine, 10 mM KH₂PO₄, 20 mM HEPES, 110 mM sucrose, and 0.25 g/L BSA, pH 7.2) containing 20 μg/mL JC-1 dye. (488ex/530em) and (533ex/590em) spectra were monitored via plate reader at 25° C. To measure membrane potential at steady-state ATP production stimulated through complex I, and II: 5 mM glutamate plus 5 mM malate, 5 mM succinate, 1 mM ADP was added to the preparation. To determine membrane potential upon inhibited ATP synthesis 3 μM oligomycin was then added. To further control for the function of the dye 25 μM of CCCP was then added to dissipate the proton gradient. A minimum of four independent mitochondrial preps were run for statistical analysis.

### Blue-Native PAGE and in, Gel Activity Assay

Assays were performed as in Garcia et al. 2017. Mitochondria were purified from 10 thoraces of 7-10 day old male flies and BN-PAGE was performed using NativePAGE gels from Life Technologies, following the manufacturer's instructions. Mitochondria were suspended in native PAGE sample buffer (Life Technologies) supplemented with 1% digitonin and protease inhibitors, incubated on ice for 20 min and centrifuged at 20,000 g for 30 min at 4° C., the supernatant was recovered, G-250 (Life Technologies) sample additive and Native PAGE Sample Buffer was added before loading onto 3-12% pre-cast Bis-Tris Native PAGE gels (Life Technologies). Electrophoreses was performed using the Native PAGE Running buffer (as anode buffer, from Life technologies) and the Native PAGE Running buffer containing 0.4% Coomassie G-250 (cathode buffer). Protein complexes were revealed by staining with Novex Colloidal Blue staining kit (Life Technologies). Complex I activity in native gels was performed by incubating gels in 0.1 mg/mL NADH, 2.5 mg/mL Nitrotetrazolium Blue Chloride, 5 mM Tris-HCl (pH 7.4) overnight at room temperature. Gels were imaged using a BioRad station and densitometry was performed in ImageJ.

### Circadian Locomotor Activity

Post-eclosion, flies were reared in a humidified, temperature-controlled incubator with 12-hour light-dark cycle at 25° C. 7 Day old adults, were anesthetized under light CO₂ and placed individually into Drosophila Activity Monitor (DAM) tubes, containing standard food. Flies were monitored using a DAM2 system (Trikinetics) for 3 days in 12 hour light-Dark cycle (LD) for circadian entrainment, followed by 6 days of constant darkness (DD) to assess free running locomotor activity. ClockLab (Coulbourn Industries) was used to determine the rhythmicity and period of the fly populations in constant darkness. A minimum of 16 flies per genotype were used for each experiment.

### TMRE/MitoSOX Intestinal Staining

Flies were anesthetized on ice and intestines were dissected in cold Schneider's Medium (Thermo Fisher Scientific). Intestines were then incubated in 75 nM of TMRE (Thermo Fisher Scientific) in Schneider's Medium, for 10 min at room temperature. Samples were rinsed three times for 30 sec with a solution consisting of 20 nM of TMRE Schneider's Medium. For MitoSOX staining dissected intestines were immersed in 50 μM MitoSOX Red (Invitrogen) in Schneider's Medium for 5 min at room temp, then washed three times for 30 sec in Schneider's Medium. MitoSOX or TMRE samples were quickly mounted in Schneider's Medium containing 1.5 μg/mL Hoechst stain. Intestines were imaged within 20 min on a Ziess LSM800 confocal microscope. Z-stacks spanning the entire posterior midgut were taken. Quantification of TMRE or MitoSOX was performed using ImageJ in which mean fluorescent intensity values were quantified. TMRE foci were quantified using the ImageJ (Schneider et al. 2012) local maxima tool, using identical thresholding for all images. Foci number were normalized to the number of nuclei in the measured field of view. Quantification of MitoSOX output of ISCs/EBs was determined by mean intensity from individual Z-planes with regions of interest defined by esgGFP expression, cell size, and basal location within the intestinal epithelium. Mean intensity of MitoSOX fluorescence levels in all ISCs/EBs were averaged for each individual intestine. A minimum of 9 midguts were used for each genotype or drug treatment.

### Phospho-Histone H3 Immunostaining

Briefly, flies were anesthetized on ice and intestines were dissected in cold PBS. Samples were then fixed in PBS+0.1% Triton X-100 containing 4% paraformaldehyde at room temperature for 30 minutes and rinsed three times in PBS+0.1% Triton X-100 for 10 minutes at room temperature. Blocking was performed in 5% BSA in PBS+0.1% triton X-100 for one hour at room temperature. Primary antibody, anti-phospho Histone H3 (S10) (Cell Signaling), was added 1:250 in 5% BSA in PBS+0.1% triton X-100 and incubated overnight at 4° C. rotating. After washing three times in PBS+0.1% Triton X-100 secondary antibody, anti-rabbit AlexaFluor-488 (Invitrogen) was added 1:250, and 1.5 μg/mL Hoechst stain (Thermo) in 5% BSA in PBS+0.2% triton X-100 and incubated overnight at 4° C. rotating. After washing, intestines were then mounted in Vectashield mounting medium (Vector Labs) and Imaged using Zeiss Ziess LSM800. pHH3 positive cells were quantified using the ImageJ (Schneider et al. 2012) local maxima tool, with identical thresholding for all images. pHH3 numbers were normalized to the area of the posterior midgut imaged. A minimum of 10 intestines were used for each quantification.

### Ese^{ts}>UAS-Notch-RNAi Intestine Staining

Ese^{ts}>UAS-Notch-RNAi flies were reared at 18° C. Upon eclosion flies were collected, sexed and placed on media containing 0.05 mM 2,4 DNP, or ethanol vehicle food at 30° C. Time-points of day 0, 8, and 16 days were taken. Intestines were dissected, fixed, and stained as above pHH3 protocol, with anti-GFP (cell signaling) and 1.5 μg/mL Hoechst stain. Quantification of GFP area was performed by using the ImageJ count particle tool in the GFP channel normalized to total area of the posterior midgut that was imaged. 7-13 guts were used for each condition and timepoint.

### Statistical Analysis

Prism7 (GraphPad) was used to perform the statistical analysis Significance is expressed as p values (ns=p>0.05, *=p<0.05, **=p<0.01, *** =p<0.001). For two group comparisons unpaired, two-tailed t-test was used, when data met criteria for parametric analysis (normal distribution and similar variance). Mann-Whitney U-test was used in case of non-parametric analysis. For more than two group's comparison ANOVA with Bonferroni, or Tukey's post-hoc test was performed. Kruskal-Wallis with Dunn's post-hoc for data that was not distributed appropriately for the parametric comparisons. For comparison of survival curves, Log-rank (Mantel-Cox) test was used.

### Example 2

### Loss of the Repressive Arm of the Transcriptional Circadian Clock Extended Lifespan Independent of Dietary Protein Restriction and Reduced Insulin-like Signaling

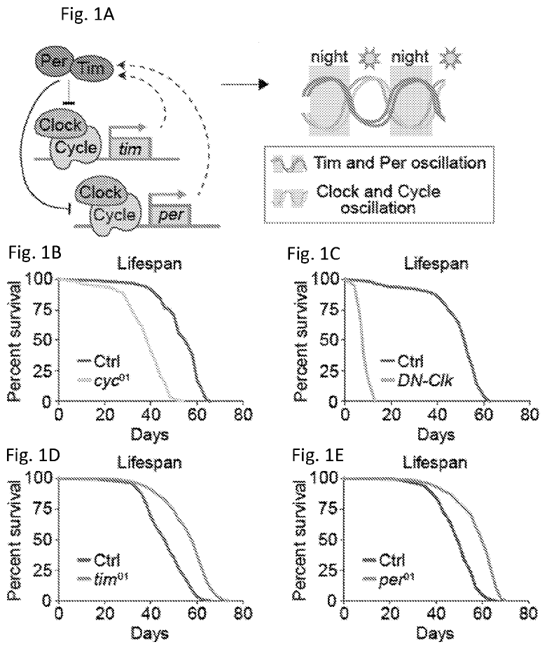

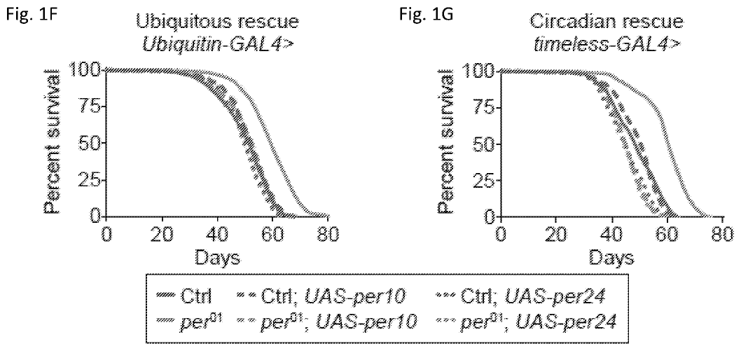

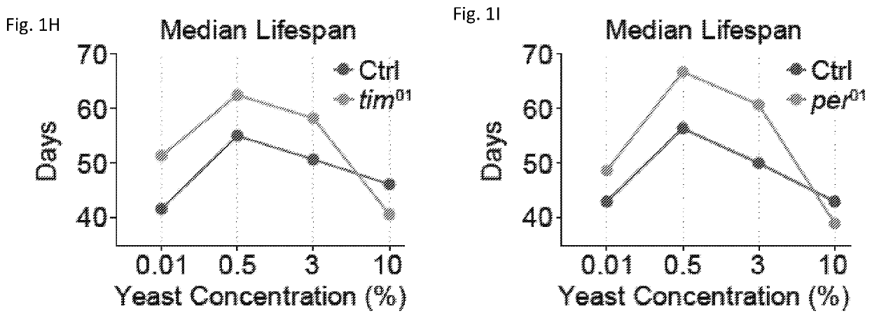

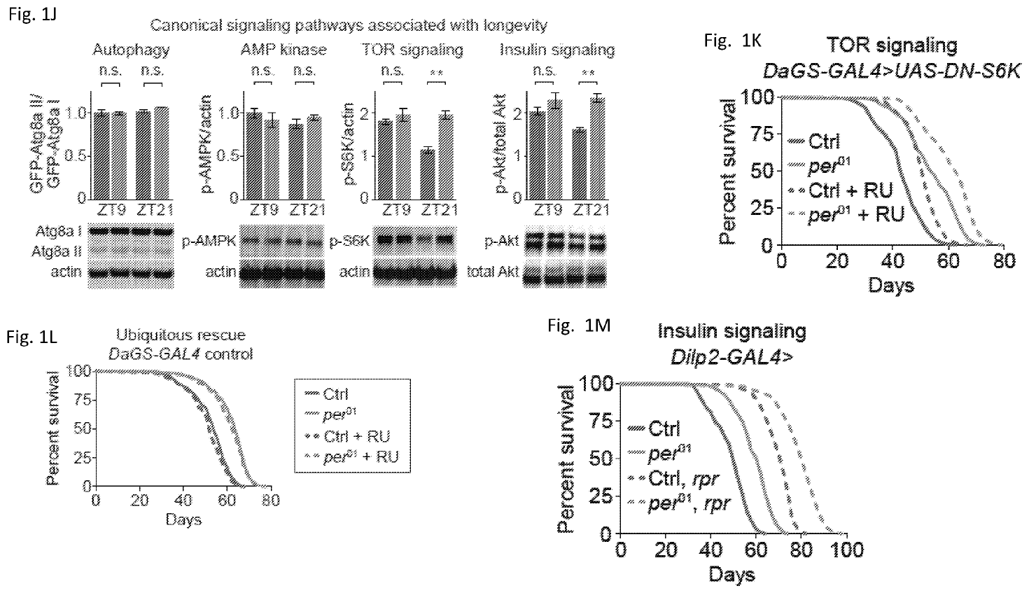

To investigate how loss of specific circadian regulators influence aging, the lifespans of four established arrhythmic Drosophila mutants (FIG. 1A): three genomic mutants, cycle (cyc⁰¹); period (per⁰¹); and timeless (tim⁰¹); and flies ubiquitously expressing a dominant-negative form of Clock (DN-Clock) (Tanoue et al. 2004) were investigated. Consistent with other reports, functional disruption of the circadian transcriptional activators Cycle or Clock shortened lifespan relative to controls (specifically males, FIGS. 1B, 1C) (Dubrovsky et al. 2010; Vaccaro et al. 2017). In contrast, functional disruption of the circadian transcriptional repressors Per and Tim significantly increased lifespan (15-20%) relative to controls; this appeared to be a male-specific effect (FIGS. 1D, 1E). To confirm that this lifespan extension was due to the loss of Per protein, Per expression was restored using the UAS-GAL4 system (Brand and Perrimon 1993). Expressing either of two independent period transgenes using either the timeless-GAL4 driver or the ubiquitin-GAL4 driver in the per⁰¹ null background reverted per mutant lifespan to that of control animals (FIGS. 1F, 1G). Thus, loss of Per expression extends lifespan.

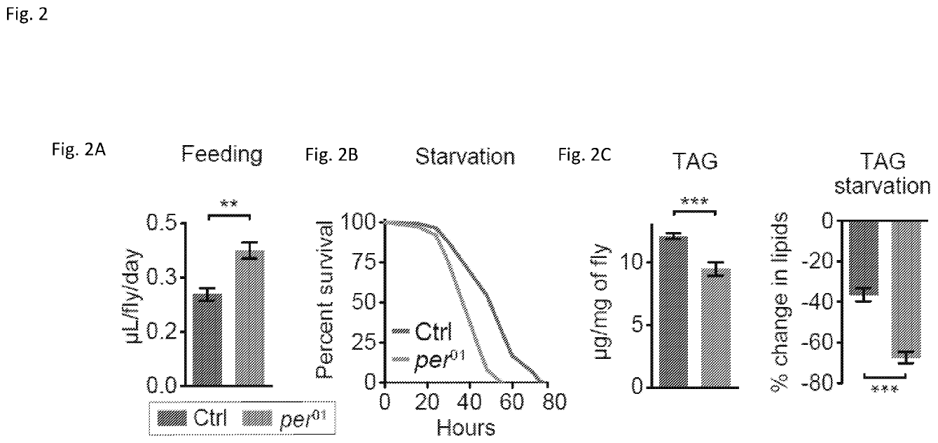

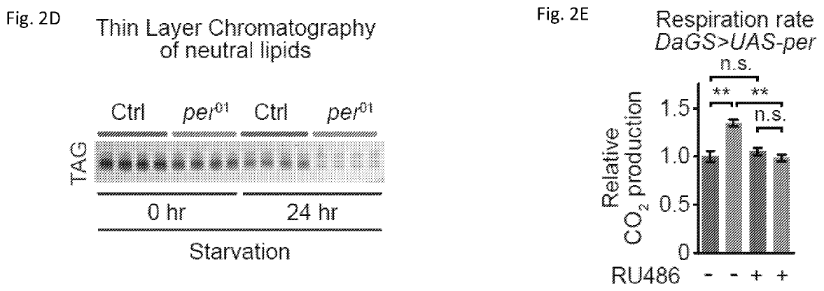

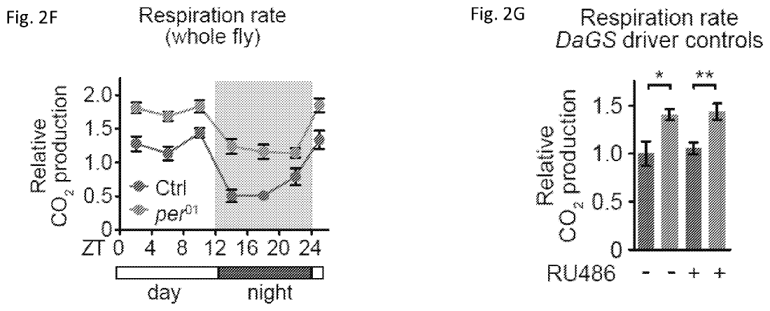

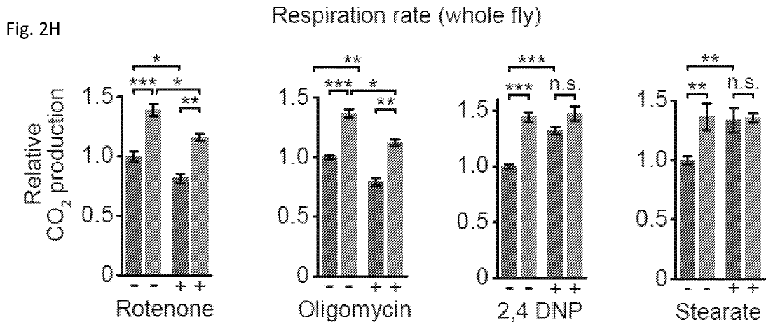

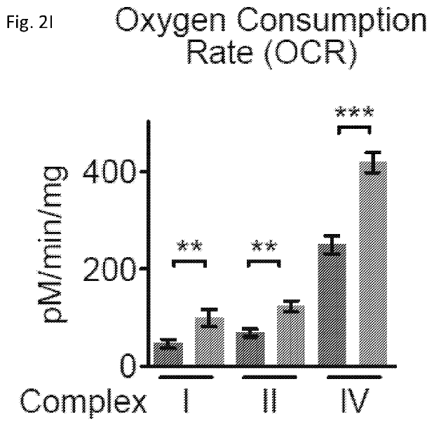

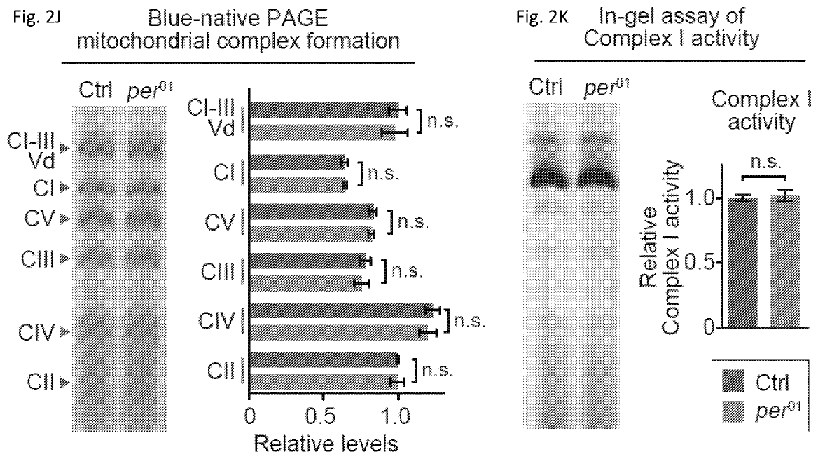

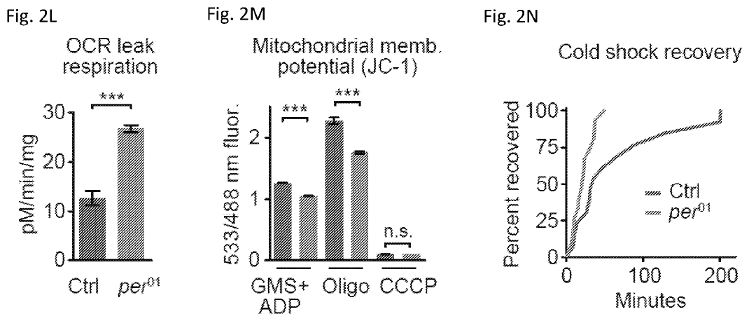

The classic method of lifespan extension is dietary restriction (DR). It has been previously shown by the inventors that per⁰¹ and tim⁰¹ mutants are not naturally diet-restricted—that is, these mutants eat more, not less, than controls (Allen et al. 2016; see also FIG. 2A). However, loss of Per and Tim might mimic physiological changes associated with DR. If so, DR should not further extend the lifespan of male per⁰¹ and tim⁰¹ mutants. In Drosophila, DR-mediated lifespan extension was accomplished by titration of protein (yeast extract). To test their response to DR, per⁰¹ and tim⁰¹ null mutants and controls were fed four different concentrations of yeast extract (YE): 0.01% (low), 0.5% (DR), 3% (standard), and 10% (high). As showed previously for female per⁰¹ and tim⁰¹ mutants (Ulgherait et al. 2016), male per⁰¹ and tim⁰¹ mutants exhibited DR-induced lifespan extensions similar to controls and lived longer than controls on most dietary protein concentrations (FIGS. 1H, 1I). The lifespan of these circadian mutants was similar to that of control animals only at very high yeast concentrations (10%), which shortens lifespan. Thus, the extended longevity of per⁰¹ mutants appeared to be independent of dietary restriction.

It was next tested if per⁰¹ mutants exhibited canonical changes in DR-associated mechanisms of longevity, including: decreased insulin signaling, measured by decreased phosphorylation of Akt protein (p-Akt); decreased TOR signaling, measured by decreased phosphorylation of S6K; and increased autophagy, measured by increased lipidation of Atg8. Surprisingly, per⁰¹ males were atypical for all of these hallmarks of longevity and, at some points in the circadian cycle, exhibited the opposite phenotype as predicted for long-lived mutants (FIG. 1J).

Genetic manipulations were used to further determine if inhibition of TOR signaling or inhibition of insulin-like-signaling (ILS) are responsible for the longevity of per⁰¹ mutants. To test inhibition of TOR signaling, TORCl signaling in adulthood was ubiquitously suppressed by inducible (RU486-mediated) overexpression of a dominant-negative form of the downstream kinase S6K, which is known to extend lifespan (Kapahi et al. 2004). Inhibition of TORCl signaling extended the lifespans of both control animals and per⁰¹ mutants to a similar magnitude (FIG. 1K), suggesting that the longevity of per⁰¹ mutants was independent of TORCl inhibition. Feeding RU486 to either controls or per⁰¹ mutants lacking the UAS-transgene had no influence on lifespan (FIG. 1L).

To test inhibition of insulin-like-signaling, partial genetic ablation of insulin-producing cells (IPCs) in the fly brain was performed using the pro-apoptotic gene reaper (Broughton et al. 2005). This extended the lifespans of both control animals and per⁰¹ mutants to a similar magnitude (FIG. 1M), suggesting that per⁰¹-associated lifespan extension is independent of insulin-like signaling inhibition.

Taken together, these results suggested that the longevity phenotype of per⁰¹ males is not due to canonical longevity mechanisms but due to a different, independent pathway.

### Example 3

### Period Mutants Exhibited a High Metabolic Rate Due to Mitochondrial Uncoupling

As metabolism and lifespan have been consistently linked, it was set out to investigate the metabolism of long-lived per⁰¹ mutants. As shown in the inventors' previous work (Allen et al. 2016), per⁰¹ males exhibited hyperphagia (increased feeding), decreased starvation resistance, low levels of lipid storage, and increased starvation-induced lipid utilization relative to controls (FIGS. 2A-2D).

Because per⁰¹ mutants eat more but are leaner than controls, hyperactive metabolic rate was tested by measuring CO₂ production. Consistent with previous characterization, per¹ mutants produced more CO₂ throughout the circadian cycle relative to controls, which was reverted by exogenous Per expression (FIGS. 2E and 2F); respiration rate was not affected in RU486 feeding controls (FIG. 2G).

Respiration rate is significantly affected by the mitochondrial function of oxidative phosphorylation. To determine if increased oxidative phosphorylation caused this increased metabolic output, flies were fed sub-lethal doses of mitochondrial complex inhibitors: rotenone, which blocks complex I of the mitochondrial electron transport chain, or oligomycin, which blocks complex V, the F₀F₁ ATP synthase. While these compounds inhibited the CO₂ output of both control and per⁰¹ animals to a similar degree, per⁰¹ flies still had higher respiration rates than controls (FIG. 2H). This suggested that their increased respiration is actually independent of mitochondrial ATP synthesis and instead implicated a different mitochondrial function, such as mitochondrial uncoupling, as the underlying mechanism.

Mitochondrial uncoupling increases respiration due to dissipation of the proton gradient and uncoupling of oxidative phosphorylation from ATP synthesis, creating futile cycles of respiration and generating heat, as in mammalian brown fat. To test the effects of mitochondrial uncoupling on respiration, per⁰¹ mutants and control flies were fed two different mitochondrial uncoupling compounds: DNP (2,4-dinitrophenol), a proton ionophore that dissipates the proton gradient; and stearic acid, which induces mitochondrial uncoupling by activating endogenous uncoupling protein (UCP) activity. Treatment with either drug increased the respiration of control flies to levels similar to those of per⁰¹ mutants but did not increase the respiration of per⁰¹ mutants (FIG. 2H). These results suggested that the increased respiration of per⁰¹ mutants was due to increased mitochondrial uncoupling and that per⁰¹ mutants may already be maximally uncoupled.

Next, to directly test whether per⁰¹ mutants were mitochondrially uncoupled relative to controls, mitochondrial function was assessed in vitro. Mitochondria from per⁰¹ mutants and controls were purified and measured for O² consumption. per⁰¹ mutant mitochondria exhibited increased O² consumption by electron transport chain (ETC) complexes I, II, and IV (FIG. 2I). This increased O² consumption was not due to increased mitochondrial ETC protein abundance or increased enzymatic activity (FIGS. 2J and 2K). Instead, per⁰¹ mitochondria exhibited two critical hallmarks of mitochondrial uncoupling relative to control mitochondria: increased leak respiration, measured by higher oxygen consumption after oligomycin treatment (FIG. 2L); and decreased membrane potential, or disrupted proton gradient, measured by the membrane potential dye sensor JC-1, both during steady-state ATP generation and after inhibition by oligomycin (FIG. 2M).

Finally, to assess heat generation, another hallmark of mitochondrial uncoupling, cold-shock recovery assays was performed on per⁰¹ mutants and control animals (FIG. 2N). After one hour of cold shock at 4° C., per⁰¹ mutants recovered significantly faster than controls, suggesting that per⁰¹ mutants may generate more heat than controls.

Thus, loss of Per protein increased mitochondrial uncoupling.

### Example 4

### Endogenous Uncoupling Protein UCP4C was Necessary for Period Mutant Lifespan Extension and Sufficient to Extend Wild-Type Lifespan

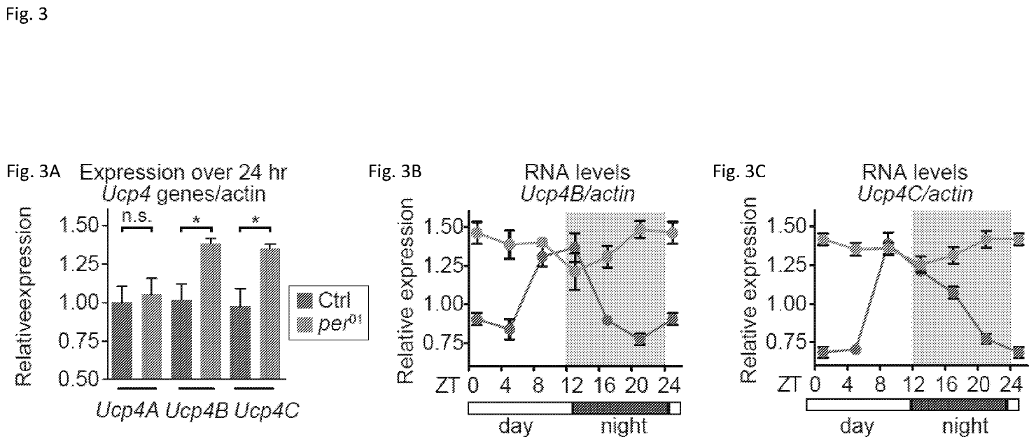

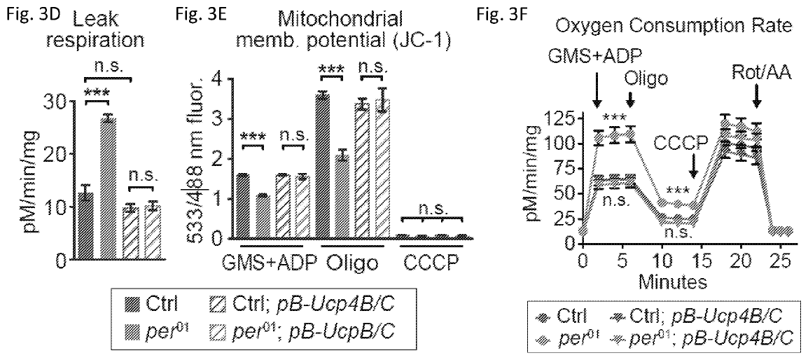

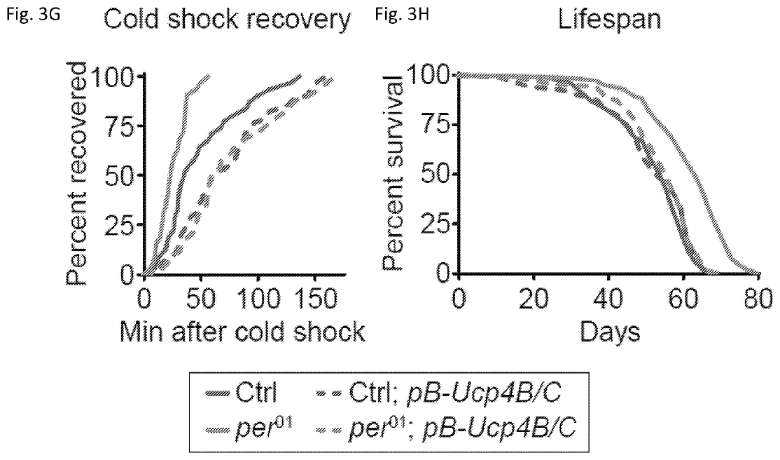

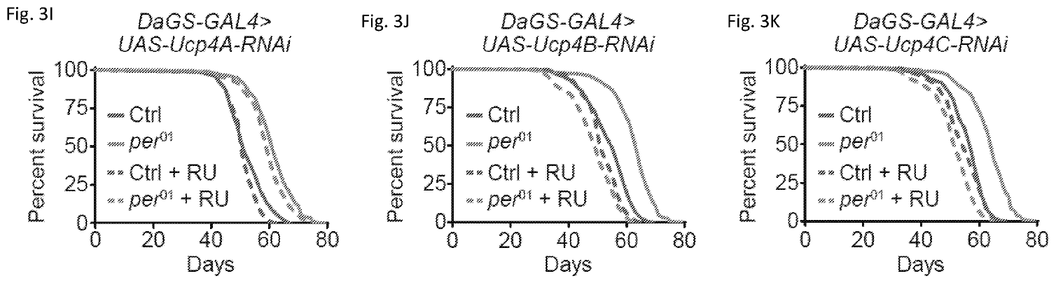

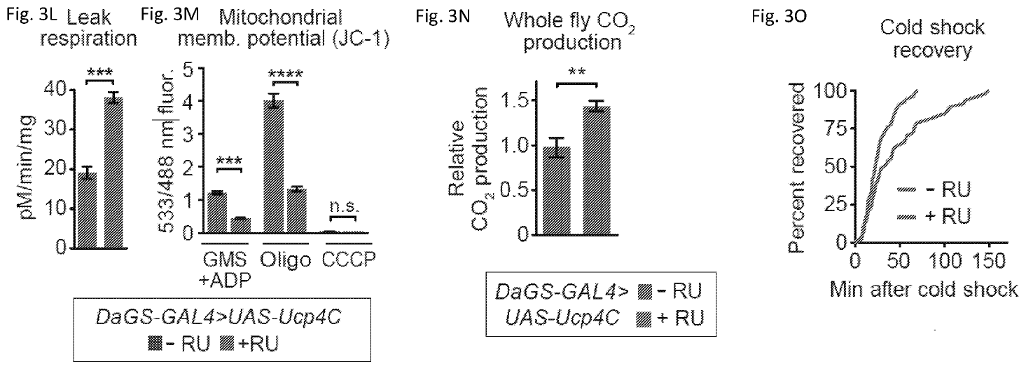

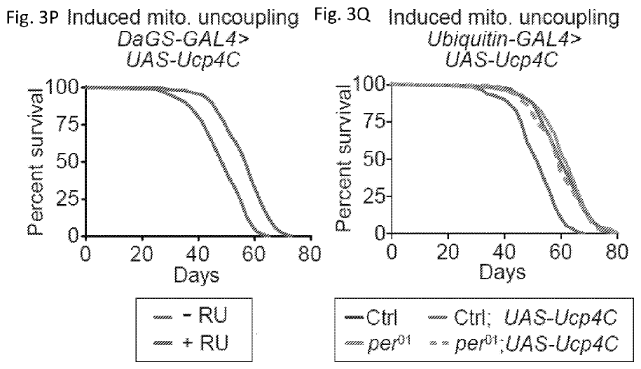

To determine if this increased mitochondrial uncoupling is required for the lifespan extension of per⁰¹ mutants, the expression of endogenous proteins was genetically manipulated to cause mitochondrial uncoupling. Like many animals, Drosophila can undergo mitochondrial uncoupling by induction of uncoupling proteins (UCPs), including UCP4A, B and C (Da-Re et al. 2014). Of these, expression of Ucp4B and Ucp4C was found to be circadian-regulated in control flies and constitutively high in per⁰¹ mutants (FIGS. 3A-C). To test the role of UCPs in the metabolic phenotype observed in per⁰¹ mutants, expression of both UCP4B/C proteins was disrupted using a mutant containing a piggyBac transposon in the intergenic region between these two closely linked Ucp4 genes. Inhibition of UCP4B/C expression reverted all of the mitochondrial uncoupling phenotypes of per⁰¹ mutants to control levels: mitochondrial leak respiration (FIG. 3D); mitochondrial membrane potential (FIG. 3E); mitochondrial oxygen consumption rate (FIG. 3F); and whole-animal cold shock recovery rates (FIG. 3G). Inhibition of UCP4B/C expression not only reverted mitochondrial uncoupling but also reverted per⁰¹ lifespan to that of controls, suggesting that mitochondrial uncoupling caused the lifespan extension (FIG. 3H). To inhibit uncoupling proteins by an orthogonal mechanism, RNAi-mediated knockdown of UCP4A, B, and C in adulthood was performed throughout the whole body in both per⁰¹ mutants and controls. Consistent with the mutant analysis, knockdown of UCP4B or C, but not UCP4A, reverted per⁰¹ lifespan to that of control animals (FIGS. 3I-3K). Thus, Ucp4B/C expression is necessary for the longevity and metabolic phenotypes of per⁰¹ mutants.

Finally, to determine if increased uncoupling protein expression was sufficient to extend the lifespan of wild-type animals, UCP4C was ubiquitously overexpressed during adulthood using RU486 feeding. Flies overexpressing UCP4C showed mitochondrial uncoupling phenotypes very similar to per⁰¹ mutants: increased mitochondrial leak respiration (FIG. 3L); decreased mitochondrial membrane potential (FIG. 3M); increased CO₂ production (FIG. 3N); and faster cold-shock recovery (FIG. 3O). Most importantly, overexpression of UCP4C extended the lifespan of otherwise wild-type flies to the same extent as per⁰¹ mutants, with no effect on per⁰¹ mutants (FIGS. 3P and 3Q). RU486 feeding alone had no effect on either metabolic or lifespan phenotypes when flies lacked the UAS transgene (results not shown).

These results suggested that UCP4C functions in the same pathway as Per to extend per⁰¹ mutant lifespan and that increased expression of the circadian-regulated mitochondrial uncoupling protein UCP4C causes lifespan extension. Taken together, the data pointed to mitochondrial uncoupling as the main circadian-regulated physiology directly responsible for the extended lifespan of per⁰¹ mutants.

### Example 5

### Lifespan Extension was Mediated by Loss of Per Specifically in the Intestine

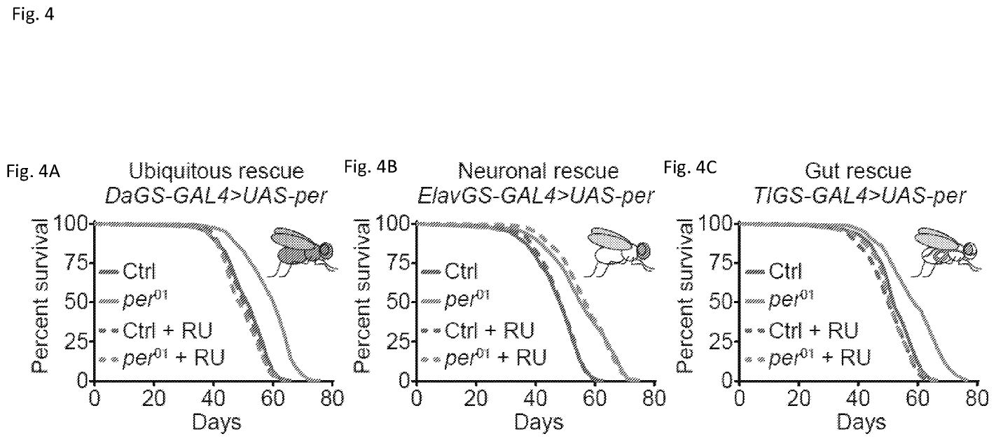

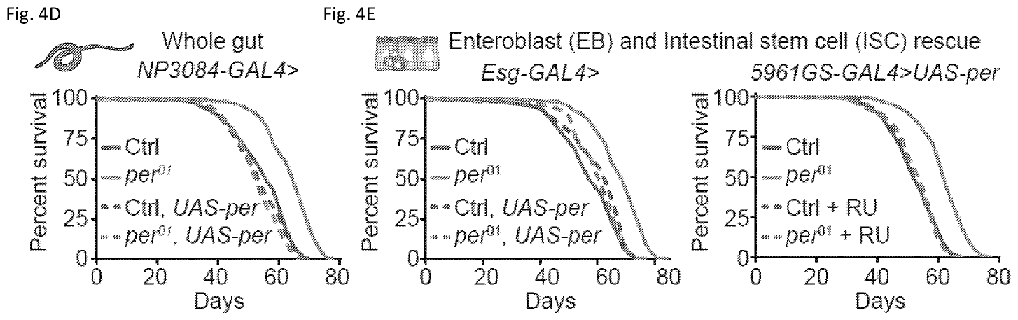

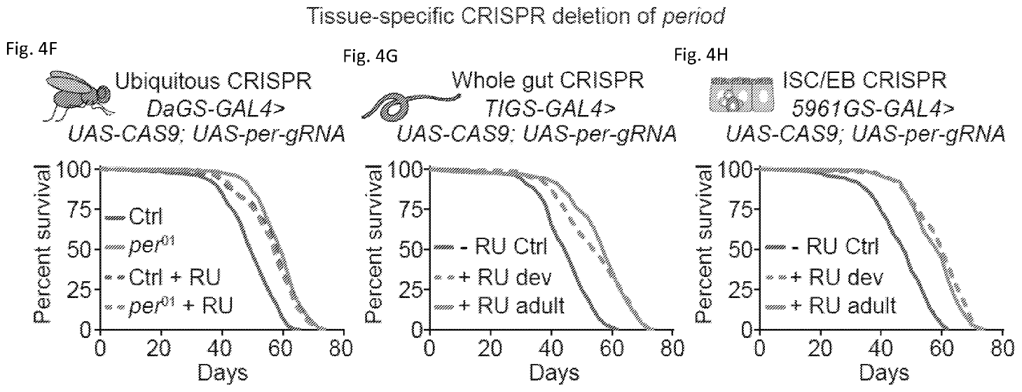

While circadian sleep/wake cycles are coordinated by a neuronal master clock in the brain, many other circadian functions are regulated by peripheral clocks in specific tissues (Allada and Chung 2010). To determine whether the master clock or a peripheral circadian clock mediates per⁰¹ longevity, period expression was rescued in different organ systems via the UAS-GAL4 system. While ubiquitous expression of Per protein during adulthood reverted the lifespan of per⁰¹ mutants to that of controls (FIG. 4A), neuronal expression of Per did not (FIG. 4B). In contrast, intestinal expression of Per in the whole intestine or specifically in intestinal stem cells (ISCs) and enteroblasts (EBs) reverted per⁰¹ lifespan to that of controls (FIGS. 4C-E). This result suggested that loss of Per in the intestine is required for lifespan extension of per⁰¹ mutants.

To directly test if loss of per in the intestine was sufficient to extend lifespan, a UAS-GAL4 based CRISPR system was used to disrupt the period gene (Port and Bullock 2016). Ubiquitous deletion of per via the daughterless GeneSwitch driver (daGS-Gal4) was sufficient to extend lifespan of control flies but not per⁰¹ mutants (FIG. 4F). Similar to per⁰¹ mutants, flies with ubiquitous disruption of period were completely arrhythmic in constant darkness (results not shown). Tissue-specific CRISPR-mediated disruption of period in either the whole intestine or specifically in ISCs/EBs during either development or adulthood was performed and found that any of these manipulations was sufficient to extend lifespan (FIGS. 4G and 4H). Disruption of period in the whole intestine or intestinal stem cells had no influence on circadian locomotor activity (results not shown). To control for CRISPR-mediated DNA damage, an unrelated gene involved in sperm storage in females (acp98AB) was disrupted (Mueller et al. 2005) using the same inducible GAL4 drivers. acp98AB disruption did not extend lifespan using the any of the tissue-specific drivers (results not shown). Similarly, RU486 feeding had no influence on control and per⁰¹ mutants lacking UAS transgenes (results not shown). CRISPR-targeted disruption of period in ISCs/EBs resulted in greater than 90% reduction in per transcript in the intestine after 30 days of induction (FIG. 4I).

Taken together, these data showed that the intestinal circadian clock plays a role in limiting lifespan in Drosophila.

### Example 6

### Upregulation of Circadian-Regulated UCP4C in the Intestine was Necessary for Loss of Per-Mediated Lifespan Extension and Sufficient to Extend Wild-Type Lifespan

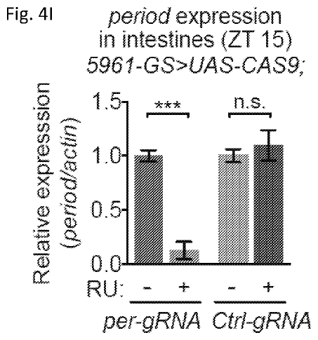

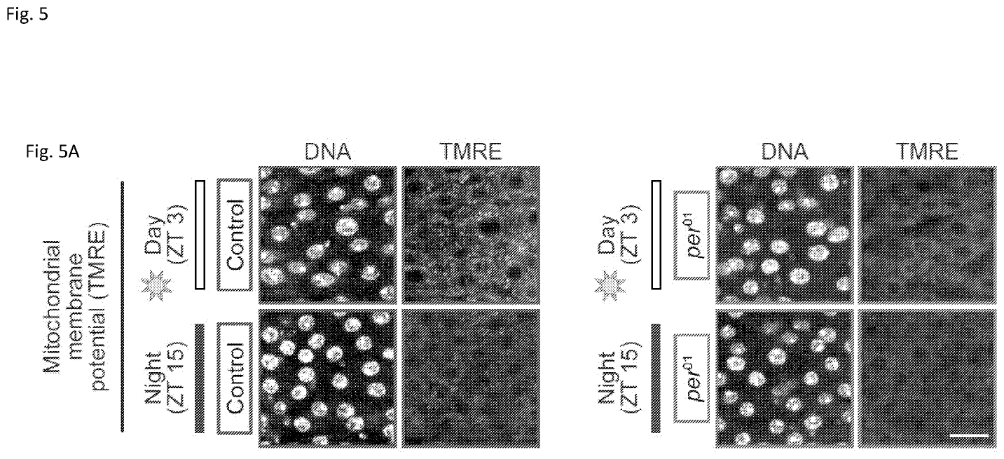

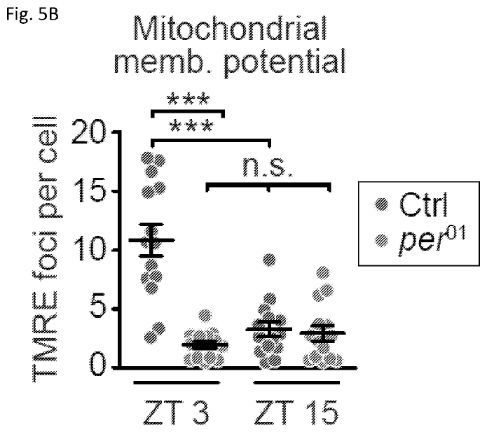

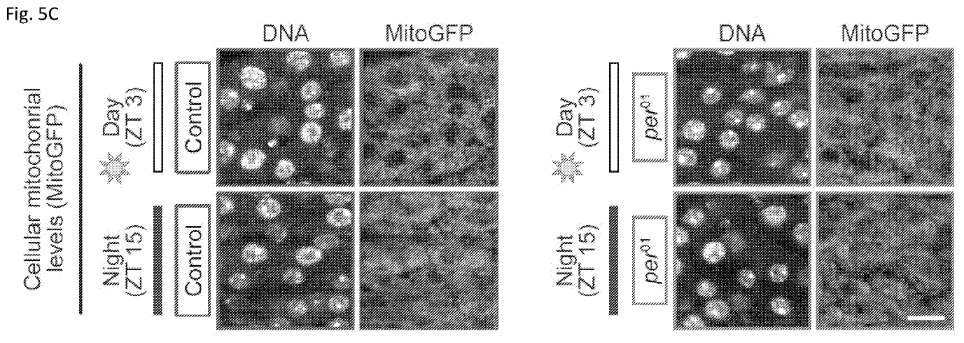

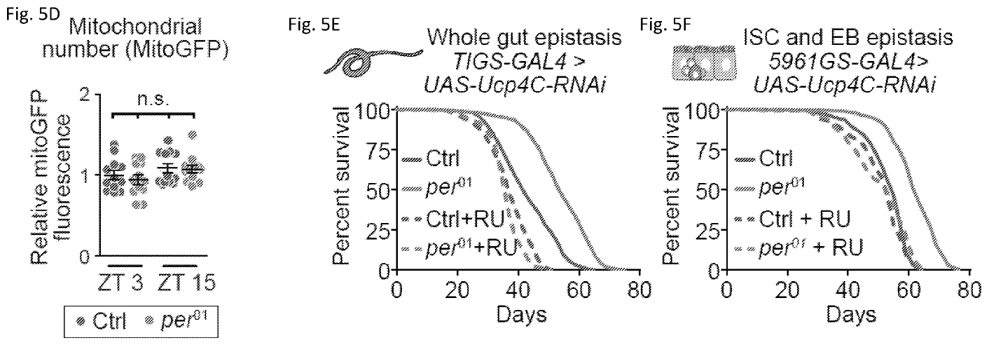

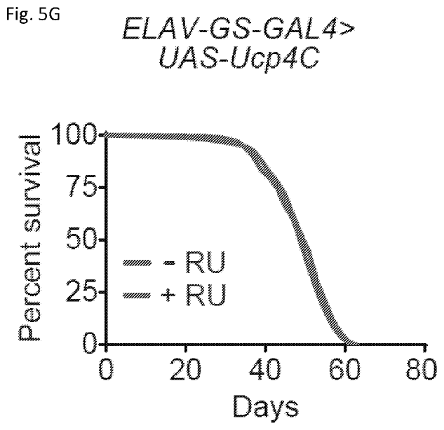

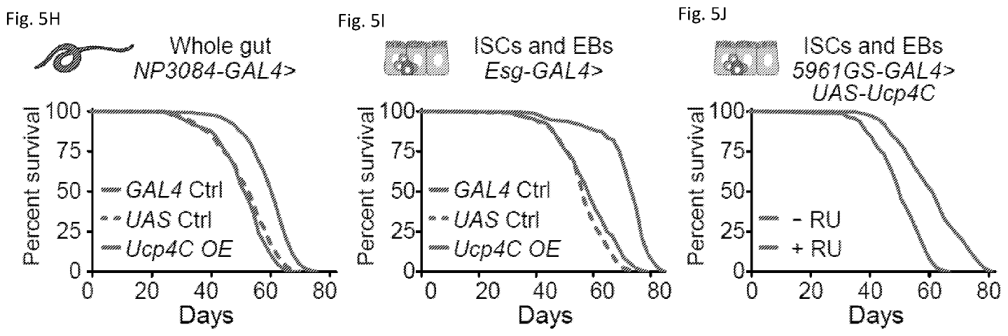

To determine if mitochondrial uncoupling is normally circadian-regulated in the intestine, mitochondrial membrane potential was measured at different times of day in dissected intestines from wild-type flies and per⁰¹ mutants, stained with the dye TMRE, which accumulates in mitochondria with higher membrane potential. Loss of membrane potential indicates mitochondrial uncoupling. In controls, intestinal mitochondrial membrane potential was indeed circadian-regulated, with higher membrane potential (lower uncoupling) during the day and lower membrane potential (higher uncoupling) at night (FIGS. 5A and 5B). Consistent with loss of circadian regulation, per⁰¹ mutants exhibited low mitochondrial membrane potential at both time points, suggesting high levels of mitochondrial uncoupling during both day and night. This phenotype was not due to changes in mitochondrial abundance, as both controls and per⁰¹ mutants had similar intestinal mitochondrial populations, as measured by mitoGFP fluorescence (FIGS. 5C and 5D). Thus, mitochondrial uncoupling in the Drosophila intestine is circadian regulated

Because it was found that ubiquitous UCP4C expression was necessary and sufficient for lifespan extension (FIG. 3), it was next tested whether intestine-specific UCP4C expression is necessary and sufficient for lifespan extension. Similar to per rescue, RNAi-mediated knockdown of UCP4C in the whole intestine or in ISC/EB populations reverted per⁰¹ lifespan to that of controls (FIGS. 5E and 5F). Moreover, while overexpression of UCP4C in the nervous system did not alter lifespan (FIG. 5G), constitutive expression of UCP4C in the whole intestine (FIG. 5H) or specifically in ISCs and EBs (FIGS. 5I, 5J) in otherwise wild-type animals lowered mitochondrial membrane potential and extended lifespan. Thus, intestinal UCP4C expression is not only necessary for the lifespan extension of per⁰¹ mutants but also sufficient to extend lifespan in otherwise wild-type Drosophila. That is, intestine-specific upregulation of a normally circadian-regulated, oscillating physiological function, mitochondrial uncoupling, extended lifespan.

### Example 7

### Loss of Period Preserved Intestinal Homeostasis via Increased Mitochondrial Uncoupling and Decreased ROS Levels

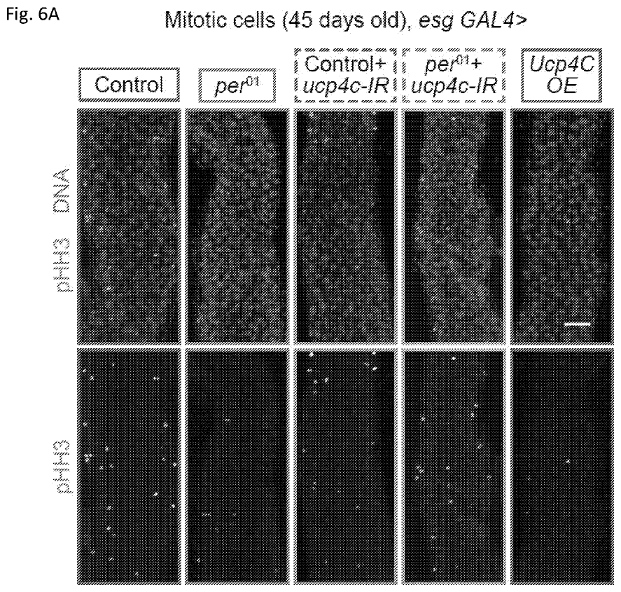

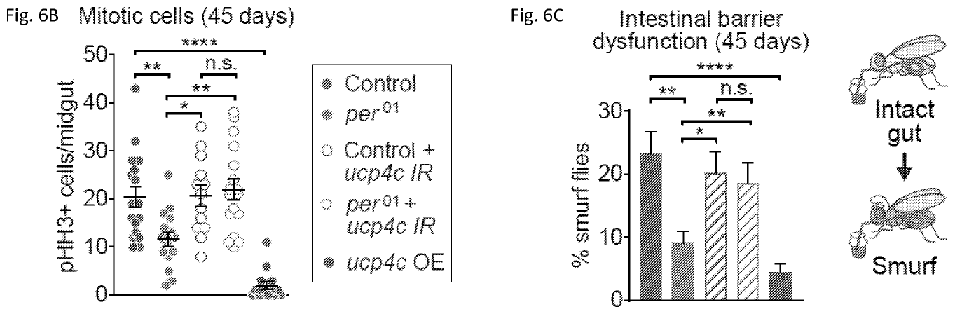

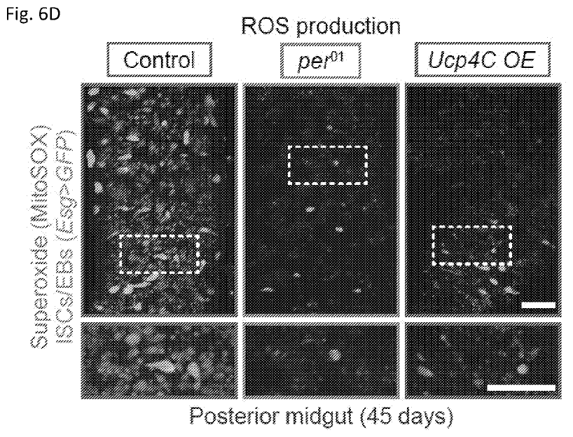

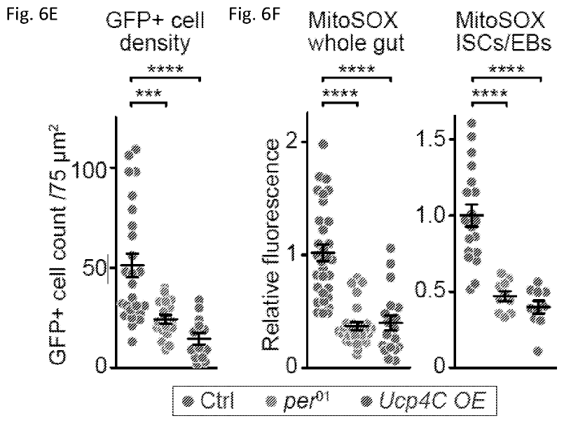

To understand the underlying mechanism by which mitochondrial uncoupling in the intestine extends lifespan, it was tested if loss of per or intestinal UCP4C overexpression delayed aging-related defects in the intestine. When flies age, they typically lose intestinal barrier function, which is also a major predictor of mortality (Rera et al. 2010; Rera et al. 2011), suggesting that lifespan can be extended by preventing aging-related intestinal barrier dysfunction. Aging-related intestinal barrier dysfunction was tested using the “smurf assay”, named after a children's cartoon, which examines for leakage of an ingested blue dye (Rera et al. 2011; Rera et al. 2012). Consistent with their extended lifespan, old per⁰¹ mutants showed a lower percentage of “smurfs” relative to controls, indicating a delay in aging-related intestinal barrier dysfunction (FIG. 6C). Moreover, this maintenance of intestinal integrity observed in per⁰¹ mutants depended on intestinal expression of UCP4C and intestinal overexpression of UCP4C alone in otherwise wild-type flies was sufficient to maintain intestinal integrity. Thus, loss of Per and increased mitochondrial uncoupling in the intestine protected against aging-related intestinal dysfunction.

As aging-related intestinal barrier dysfunction is linked to overproliferation and tissue dysplasia in the gut (Hu and Jasper 2017), it was next tested if loss of per or intestinal UCP4C overexpression delayed aging-related cellular proliferation in the gut. Per protein has previously been shown to regulate intestinal stem cell regeneration during acute intestinal stress (Karpowicz et al. 2013). To measure cellular proliferation in the intestines, aged per⁰¹ and control flies were stained for phospho-histone H3 at serine 10 (pHH3), a standard proliferative marker that labels mitotic cells (FIGS. 6A and 6B). Again consistent with delayed aging, per⁰¹ mutants showed fewer pHH3 positive cells in the gut relative to control flies. To determine if this phenotype is dependent on mitochondrial uncoupling, UCP4C was knocked down in per⁰¹ flies and controls. UCP4C knockdown reverted the number of proliferative cells in per⁰¹ flies to the higher levels seen in controls. Moreover, overexpression of UCP4C in otherwise wild-type controls was sufficient to decrease age-related overproliferation of ISCs/EBs. Thus, similar to the smurf assay and consistent with lifespan results, intestinal UCP4C expression was both necessary in per⁰¹ mutants and sufficient in otherwise wild-type controls to suppress aging-related intestinal cellular proliferation.

Intestinal dysfunction and increased aging-related cellular proliferation are closely associated with elevated Reactive Oxygen Species (ROS) production. ROS can serve as important mitogenic signals in stem cells and their differentiated progeny cells in the fly (Owusu-Ansah and Banerjee 2009); elevated ROS production in the Drosophila intestine is thought to lead to increased misdifferentiation of proliferative cells, intestinal stem cells (ISCs) and enteroblasts (EBs) (Hochmuth et al. 2011). Because mitochondrial uncoupling can restrict ROS output (Mailloux and Harper 2011), it was hypothesized that uncoupled per⁰¹ mutants have extended longevity due to decreased mitochondrial ROS production in the intestine, leading to a decrease in age-related overproliferation of esg-positive (esg+) ISC/EB cells. To test this, the intestines of aged per⁰¹ mutants, flies expressing UCP4C constitutively in ISCs and EBs, and controls with MitoSOX Red, were stained with a fluorescent mitochondrial ROS (superoxide) indicator (FIG. 6D). Both per⁰¹ mutants and intestinal UCP4C-expressing flies exhibited reduced esg+populations and decreased mitochondrial superoxide production relative to controls in the whole posterior midgut, as well as in ISC/EB populations (FIGS. 6E and 6F).

Together, these results suggested that loss of Per function and increased mitochondrial uncoupling via increased UCP4C lead to a decrease in ROS levels in the intestine.

### Example 8

### Pharmacological Reduction of ROS via Uncoupling Preserves Intestinal Homeostasis and Extends Lifespan

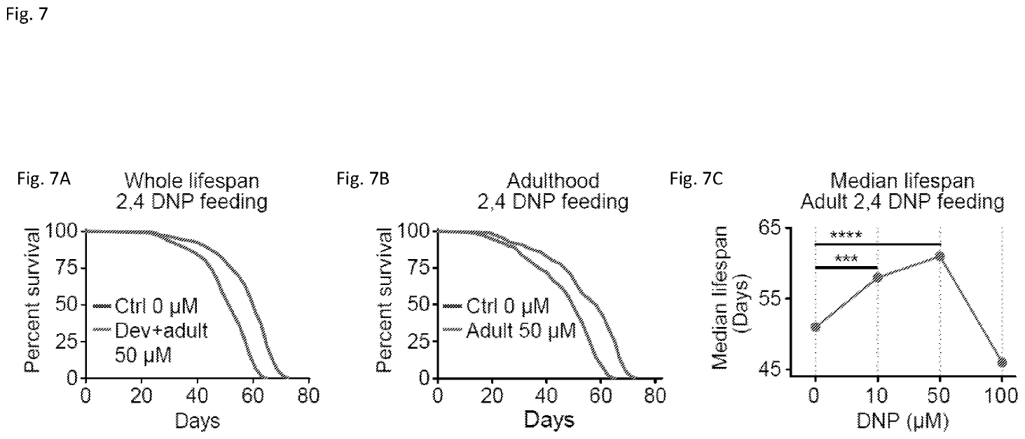

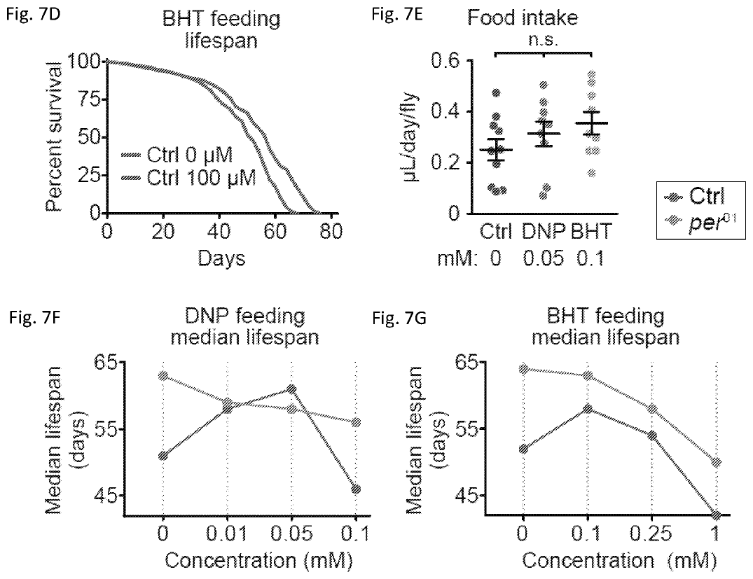

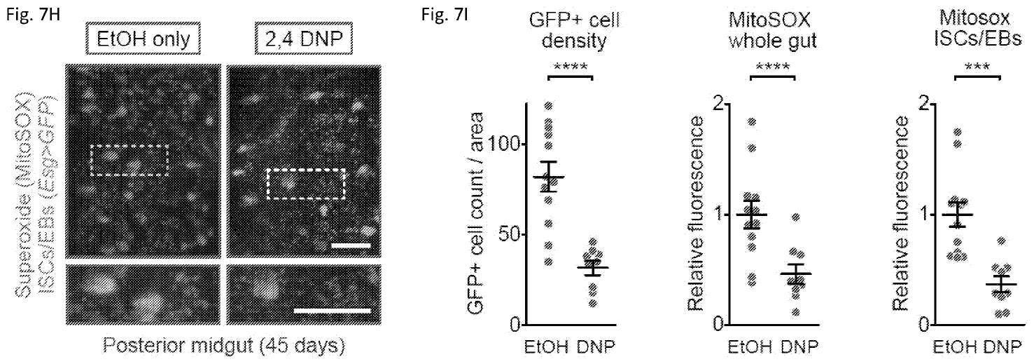

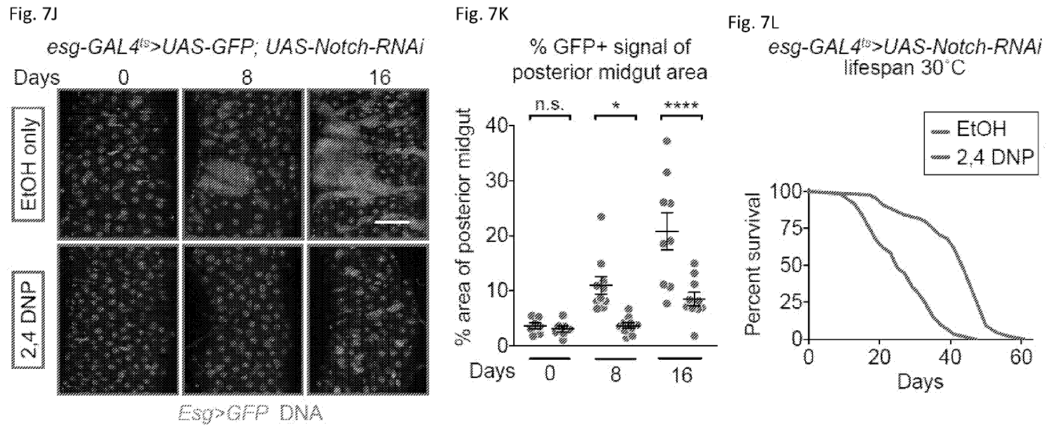

To extend the genetic experiments, pharmacological induction of mitochondrial uncoupling was tested to see if it recapitulates the intestinal phenotypes of per⁰¹ mutants and extends lifespan. Low dietary concentrations of the mitochondrial uncoupling compound DNP (2,4-dinitrophenol) or uncoupling-agent/antioxidant beta-hydroxytoluene (BHT) extended the lifespan of wild-type male flies in a dose-dependent manner within a range of physiological concentrations (FIGS. 7A-D) (Caldeira da Silva et al. 2008; Lou et al. 2007). To ensure that these drugs did not cause diet restriction, it was confirmed that DNP or BHT did not decrease feeding rate (FIG. 7E). Additionally, these drugs did not extend the lifespan of per⁰¹ mutants, suggesting that these mutants are already receiving the maximal benefit of mitochondrial uncoupling (FIGS. 7F and 7G). DNP feeding also reduced age-related esg+cell overproliferation, mitochondrial ROS output of the entire posterior midgut, and ISC/EB populations (FIGS. 7H and 7I). Thus, pharmacological induction of mitochondrial uncoupling extended lifespan and recapitulated many of the metabolic phenotypes of per⁰¹ mutants and UCP4C—expressing flies.

### Example 9

### Mitochondrial Uncoupling Slowed Tumor Progression

Changes to ISCs that alter differentiation and result in overproliferation are a common step in cancer development (Zhai et al. 2015). Notch-Delta signaling normally keeps ISCs/EBs at a homeostatic level of differentiation and division and loss of Notch specifically in ISCs and EBs thus leads to high levels of proliferation and the formation of ISC-derived intestinal tumors (Ohlstein and Spradling 2007).

To test if mitochondrial uncoupling could slow tumor progression in the gut, ISC tumors were induced by suppressing Notch signaling via RNAi, followed by feeding of 2,4 DNP (to induce mitochondrial uncoupling) or vehicle. Dietary 2,4 DNP slowed the progression of ISC tumors (FIG. 7J and 7K) and also extended the shortened lifespan of these animals (FIG. 7L). These results suggested that induction of mitochondrial uncoupling can decrease stem cell overproliferation not only during normal aging but also during induced tumorigenesis.

### REFERENCES

Allada and Chung (2010). Circadian organization of behavior and physiology in Drosophila. Annu Rev Physiol 72, 605-624.
Allen et al. (2016). period-Regulated Feeding Behavior and TOR Signaling Modulate Survival of Infection. Curr Biol 26, 184-194.
Berger et al. (2016). Mitochondrial function controls intestinal epithelial sternness and proliferation. Nature Communications 7, 13171.
Brand and Perrimon (1993). Targeted gene expression as a means of altering cell fates and generating dominant phenotypes. Development 118, 401-415.
Broughton et al. (2005). Longer lifespan, altered metabolism, and stress resistance in Drosophila from ablation of cells making insulin-like ligands. Proc Natl Acad Sci U S A 102, 3105-3110.
Brown (2016). Circadian Metabolism: From Mechanisms to Metabolomics and Medicine. Trends Endocrinol Metab 27, 415-426.
Caldeira da Silva et al. (2008). Mild mitochondrial uncoupling in mice affects energy metabolism, redox balance and longevity. Aging Cell 7, 552-560.

### Da-Re et al. (2014). UCP4C mediates uncoupled respiration in larvae of Drosophila melanogaster. EMBO Rep 15, 586-591.

Dubrovsky et al. (2010). Deficiency of circadian protein CLOCK reduces lifespan and increases age-related cataract development in mice. Aging 2, 936-944.
Epstein et al. (2001). Genome-wide responses to mitochondrial dysfunction. Mol Biol Cell 12, 297-308.
Garcia et al. (2017). Regulation of Mitochondrial Complex I Biogenesis in Drosophila Flight Muscles. Cell Rep 20, 264-278.
Grimaldi et al. (2010). PER2 Controls Lipid Metabolism by Direct Regulation of PPARγ. Cell Metabolism 12, 509-520.
Hochmuth et al. (2011). Redox regulation by Keap1 and Nrf2 controls intestinal stem cell proliferation in Drosophila. Cell Stem Cell 8, 188-199.
Hu and Jasper (2017). Epithelia: Understanding the Cell Biology of Intestinal Barrier Dysfunction. Curr Biol 27, R185-R187.
Ja et al. (2007). Prandiology of Drosophila and the CAFE assay. Proc Natl Acad Sci U S A 104, 8253-8256.
Kapahi et al. (2004). Regulation of lifespan in Drosophila by modulation of genes in the TOR signaling pathway. Curr Biol 14, 885-890.
Karpowicz et al. (2013). The circadian clock gates the intestinal stem cell regenerative state. Cell Rep 3, 996-1004.
Kenwood et al. (2014) Identification of a novel mitochondrial uncoupler that does not depolarize the plasma membrane. Molecular Metabolism 3:2 114-123
Lee et al. (2010). Disrupting Circadian Homeostasis of Sympathetic Signaling Promotes Tumor Development in Mice. PLOS ONE 5, e10995.
Lou et al. (2007). Mitochondrial uncouplers with an extraordinary dynamic range. Biochem J 407, 129-140.
Mailloux et al. (2011). Uncoupling proteins and the control of mitochondrial reactive oxygen species production. Free Radic Biol Med 51, 1106-1115.
Mueller et al. (2005). Cross-Species Comparison of Drosophila Male Accessory Gland Protein Genes. Genetics 171, 131-143.
Myant et al. (2013). ROS Production and NF-κB Activation Triggered by RACl Facilitate WNT-Driven Intestinal Stem Cell Proliferation and Colorectal Cancer Initiation. Cell Stem Cell 12, 761-773.
Ohlstein and Spradling (2007). Multipotent Drosophila Intestinal Stem Cells Specify Daughter Cell Fates by Differential Notch Signaling. Science 315, 988-992.
Owusu-Ansah and Banerjee (2009). Reactive oxygen species prime Drosophila haematopoietic progenitors for differentiation. Nature 461, 537-541. Panda (2016). Circadian physiology of metabolism. Science 354, 1008-1015.
Port and Bullock (2016). Augmenting CRISPR applications in Drosophila with tRNA-flanked Cas9 and Cpf1 sgRNAs. Nature methods 13, 852-854.
Rera et al. (2011). Modulation of longevity and tissue homeostasis by the Drosophila PGC-1 homolog. Cell Metab 14, 623-634.
Rera et al. (2012). Intestinal barrier dysfunction links metabolic and inflammatory markers of aging to death in Drosophila. Proc Natl Acad Sci USA 109, 21528-21533.
Schneider et al. (2012). NIH Image to ImageJ: 25 years of image analysis. Nat Methods 9, 671-675.
Tao et al. (2014) Niclosamide ethanolamine-induced mild mitochondrial uncoupling improves diabetic symptoms is mice. Nat. Med. 20, 1263-1269
Tanoue et al. (2004). Circadian clocks in antennal neurons are necessary and sufficient for olfaction rhythms in Drosophila. Curr Biol 14, 638-649.
Ulgherait et al. (2016). Dietary Restriction Extends the Lifespan of Circadian Mutants tim and per. Cell Metab 24, 763-764.
Vaccaro et al. (2017). Drosophila Clock Is Required in Brain Pacemaker Neurons to Prevent Premature Locomotor Aging Independently of Its Circadian Function. PLoS Genet 13, e1006507.
Wallace and Starkov (2000) Mitochondrial Targets of Drug Toxicity Ann. Rev. Pharm. Toxicol. 40:353-88
Wang et al. (2016a). Per2 induction limits lymphoid-biased haematopoietic stem cells and lymphopoiesis in the context of DNA damage and ageing. Nat Cell Biol 18, 480-490.
Wang et al. (2016b). Negative reciprocal regulation between Sirt1 and Per2 modulates the circadian clock and aging. Scientific Reports 6, 28633.
Yatsenko et al. (2014). Measurement of metabolic rate in Drosophila using respirometry. J Vis Exp, e51681.
Zhai et al. (2015). Accumulation of differentiating intestinal stem cell progenies drives tumorigenesis. Nature Communications 6, 10219.

1. A method of slowing or inhibiting tumor growth in a subject in need thereof by administering a therapeutically effective amount of an agent which activates mitochondrial uncoupling.

2. The method of claim 1, wherein the tumor is located in an organ or tissue chosen from the group consisting of: lungs; breast; liver; kidney; pancreas; esophagus; stomach; small intestine; large intestine; bones; and brain.

3. The method of claim 1, wherein the agent which activates mitochondrial uncoupling is chosen from the group consisting of substituted phenols, trifluoromethylbenzimidazoles, salicylanilides, carbonyl cyanide phenylhydrazones, fatty acids, membrane-active peptides, retinoic acid, and carotenoids.

4. The method of claim 1, wherein the agent which activates mitochondrial uncoupling is chosen from the group consisting of RU486, stearic acid, 2,4-dinitrophenol (2,4 DNP or DNP), butylated hydroxytoluene (BHT), niclosamide, nitazoxanide, and BAM15.

5. The method of claim 1, wherein the agent which activates mitochondrial uncoupling is a nucleic acid encoding an uncoupling protein or a variant, mutant, fragment, homologue, or derivative thereof.

6. (canceled)

7. The method of claim 1, further comprising administering another agent chosen from the group consisting of agents which inhibit cellular proliferation, chemotherapeutic agents and immunomodulating agents.

8. The method of claim 1, wherein the agent which activates mitochondrial uncoupling is administered to the organ or tissue in which the tumor is located.

9. (canceled)

10. (canceled)

11. A method of preventing or treating cancer in a subject in need thereof by administering a therapeutically effective amount of an agent which activates mitochondrial uncoupling.

12. The method of claim 11, wherein the cancer is located in an organ or tissue chosen from the group consisting of lungs; breast; liver; kidney; pancreas;
esophagus; stomach; small intestine; large intestine; bones; and brain.

13. The method of claim 11, wherein the agent which activates mitochondrial uncoupling is chosen from the group consisting of substituted phenols, trifluoromethylbenzimidazoles, salicylanilides, and carbonyl cyanide phenylhydrazones, fatty acids, membrane-active peptides, retinoic acid, and carotenoids.

14. The method of claim 11 wherein the agent which activates mitochondrial uncoupling is chosen from the group consisting of RU486, stearic acid, 2,4-dinitrophenol (2,4 DNP or DNP), butylated hydroxytoluene (BHT), niclosamide, nitazoxanide, and BAM15.

15. The method of claim 11, wherein the agent which activates mitochondrial uncoupling is a nucleic acid encoding an uncoupling protein or a variant, mutant, fragment, homologue, or derivative thereof.

16. The method of claim 11, wherein the agent further comprises a pharmaceutically acceptable a ligand, conjugate, vector, lipid, carrier, adjuvant or diluent.

17. The method of claim 11, further comprising administering another agent chosen from the group consisting of agents which inhibit cellular proliferation, chemotherapeutic agents and immunomodulating agents.

18. The method of claim 11, wherein the agent which activates mitochondrial uncoupling is administered to the organ or tissue in which the tumor is located.

19.-31. (canceled)

32. A method of decreasing or suppressing harmful or undesirable cellular proliferation in a subject in need thereof by administering a therapeutically effective amount of an agent which activates mitochondrial uncoupling.

33. (canceled)

34. (canceled)

35. (canceled)

36. (canceled)

37. The method of claim 32, wherein the agent which activates mitochondrial uncoupling is chosen from the group consisting of substituted phenols, trifluoromethylbenzimidazoles, salicylanilides, carbonyl cyanide phenylhydrazones, fatty acids, membrane-active peptides, retinoic acid, and carotenoids.

38. The method of claim 32, wherein the agent which activates mitochondrial uncoupling is chosen from the group consisting of RU486, stearic acid, 2,4-dinitrophenol (2,4 DNP or DNP), butylated hydroxytoluene (BHT), niclosamide, nitazoxanide, and BAM15.

39. The method of claim 32, wherein the agent which activates mitochondrial uncoupling is a nucleic acid encoding an uncoupling protein or a variant, mutant, fragment, homologue, or derivative thereof.

40. (canceled)

41. The method of claim 32, wherein the agent which activates mitochondrial uncoupling is administered to the intestines.

42.-52. (canceled)
